(19) United States
(12) Patent Application Publication
(10) Pub. No.: US 2023/0266332 A1
(43) Pub. Date: Aug. 24, 2023

(54) METHODS AND COMPOSITIONS FOR PREVENTING AND TREATING A CANCER

(71) Applicants: INSERM (INSTITUT NATIONAL DE LA SANTÈ ET DE LA RECHERCH MÉDICALE), Paris (FR); UNIVERSITÉ PARIS CITÉ, Paris (FR); Assistance Publique-Hôpitaux de Paris, Paris (FR)

(72) Inventors: Eric TARTOUR, PARIS (FR); Stephane OUDARD, PARIS (FR); Ikuan SAM, PARIS (FR); Nadine BEN HAMOUDA, PARIS (FR)

(21) Appl. No.: 18/007,248

(22) PCT Filed: Jul. 27, 2021
(86) PCT No.: PCT/EP2021/071068

§ 371 Date: Jan. 27, 2023

(30) Foreign Application Priority Data
Jul. 28, 2020 (EP) 20305866.4

(51) Int. Cl. G01N 33/574; C07K 16/28

(52) U.S. Cl. CPC G01N 33/57488; C07K 16/2875; C07K 16/2878; C07K 16/2803; G01N 2333/70578; G01N 2333/70575; G01N 2800/50; A61K 2039/507

(57) ABSTRACT

Inventors have shown that CD70 and CD27 are highly expressed in ccRCC and correlates with poor survival. Multiplex IF demonstrated that CD27+T cells interact with CD70+ tumor cells in tumor microenvironment (TME). CD27+T cells are more apoptotic than CD27−T cells in ccRCC. Elevated levels of plasma sCD27 is observed in ccRCC patients and correlates with CD27−CD70 interaction in situ. Their study demonstrates that CD27−CD70 interaction contributes to the release of sCD27 in peripheral blood in ccRCC, indicating that sCD27 is a potential biomarker. The apoptosis of CD27+T cells suggests the deleterious effect of CD27−CD70 interaction in T cell response. Therefore, CD27/CD70 is a promising therapeutic target in ccRCC. Accordingly, the invention relates to a method for determining the interaction between CD27 and CD70 by determining the level of soluble CD27 (sCD27) in a biological sample and to method of targeting CD27/CD70 interaction to treat a cancer or metastatic cancer.

### FIELD OF THE INVENTION

The invention is in the field of oncology. More particularly, the invention relates to methods and composition for preventing and treating a cancer or a metastatic cancer.

### BACKGROUND OF THE INVENTION

Renal cell carcinoma (RCC) accounts for 3-4% of all cancers with more than 300,000 new cases diagnosed worldwide and 140,000 deaths yearly (Capitanio et al., 2019). Clear cell renal cell carcinoma (ccRCC) represents 70-75% of histological subtype of RCC and is characterized by the inactivation of the von Hippel-Lindau (VHL) tumor suppressor gene, which leads to the stabilization and accumulation of hypoxia-inducible factor-2 (HIF-2). HIF target genes regulate angiogenesis, glycolysis and apoptosis (Gossage et al., 2015).

Accordingly, uncontrolled activation of HIF explained why ccRCC tumors are rich in lipids, glycogens and are highly vascular. Localized RCC can be treated with partial or radical nephrectomy. However, approximately 30% of patients with localized ccRCC eventually develop metastases (Hsieh et al., 2017). Given the nature of RCC, anti-angiogenic therapies targeting vascular endothelial growth factor (VEGF) signaling axis (sunitinib, bevacizumab, axitinib) and mammalian target of rapamycin (mTOR) inhibitor (everolimus, temsirolumus) have been approved for the treatment of metastatic RCC (Hsieh et al., 2017).

CD70, a member of the tumor necrosis factor superfamily (TNFSF), is typically present on activated memory B and T lymphocytes, natural killer (NK) cells and mature DCs (Borst et al., 2005; Nolte et al., 2009). CD70 expression is necessary for an effective immune response, such as T cell activation and proliferation and memory cell generation, B cell activation and differentiation. CD70 expression is necessary for an effective immune response, such as T cell activation and proliferation and memory cell generation, B cell activation and differentiation. (Garcia et al., 2004) (Arens et al., 2004). Interestingly, CD70 is aberrantly found in hematological and solid tumors, especially in ccRCC (about 80%) (Law et al., 2006; Ruf et al., 2015). Moreover, high CD70 expression in ccRCC is associated with decreased survival (Jilaveanu et al., 2012). The consequences of this interaction on immune cells in the TME are still unclear.

CD27, the ligand of CD70, is a co-stimulatory molecule and belongs to TNF receptor family (Buchan et al., 2018b; Camerini et al., 1991). CD27 is constitutively expressed on naïve T cells and central-memory T cells, but is downregulated on effector T cells (Mahnke et al., 2013). CD27−CD70 axis is critical for T cell activation during the priming phase. CD27−CD70 interaction triggers a series of additional co-stimulatory signals, which results in the expansion and differentiation of memory and effector T cells (Nolte et al., 2009). Studies elucidating CD27−CD70 interaction in solid tumor is rare. One study suggested that RCC-expressed CD70 was driving RCC tumor infiltrating lymphocytes (TILs) to terminal differentiation (Wang et al., 2012). Another two in vitro studies suggested that CD27−CD70 interaction induces apoptosis in T cells, which serve as an immune escape mechanisms of CD70-expressing glioblastoma and ccRCC (Diegmann et al., 2006; Wischhusen et al., 2002). Finally, soluble CD27 (sCD27) is derived from proteolytic cleavage of the transmembrane molecule on activated T cells after CD27−CD70 interaction (Nolte et al., 2009). The role of this soluble receptor and its prognostic value in solid tumors is not known.

Accordingly, identifying the role of soluble CD27 and its interaction with CD70 in solid tumors will allow to understand and find new tools to treat solid tumor.

### SUMMARY OF THE INVENTION:

The invention relates to a method for determining the interaction between CD27 and CD70 comprising the steps of: i) determining the level of soluble CD27 (sCD27) in a biological sample; ii) comparing the level of sCD27 quantified at step i) with its corresponding predetermined reference value and iii) concluding that there is an interaction between CD27 and CD70, when the level of sCD27 is higher than its corresponding predetermined reference value or concluding that there is not any interaction between CD27 and CD70, when the level of sCD27 is lower than its corresponding predetermined reference value. In particular, the invention is defined by claims.

### DETAILED DESCRIPTION OF THE INVENTION

Inventors have shown that CD70 and CD27 are highly expressed in ccRCC and correlates with metastatic disease. Multiplex IF demonstrated that CD27⁺T cells interact with CD70⁺ tumor cells in tumor microenvironment (TME). CD27⁺T cells are more apoptotic than CD27⁻T cells in ccRCC. Elevated levels of plasma sCD27 is observed in ccRCC patients and correlates with CD27−CD70 interaction in situ. Their study demonstrates that CD27−CD70 interaction contributes to the release of sCD27 in peripheral blood in ccRCC, indicating that sCD27 is a potential biomarker. The apoptosis of CD27⁺T cells suggests the deleterious effect of CD27−CD70 interaction in T cell response. Therefore, CD27/CD70 is a promising therapeutic target in ccRCC.

### Method for Determining the Interaction Between CD27 and CD70

Accordingly, in a first aspect, the invention relates to a method for determining the interaction between CD27 and CD70 comprising the steps of: i) determining the level of soluble CD27 (sCD27) in a biological sample; ii) comparing the level of sCD27 quantified at step i) with its corresponding predetermined reference value and iii) concluding that there is an interaction between CD27 and CD70, when the level of sCD27 is higher than its corresponding predetermined reference value or concluding that there is not any interaction between CD27 and CD70, when the level of sCD27 is lower than its corresponding predetermined reference value.

In a particular embodiment, the method according to the invention is suitable to predict the dysfunction of intratumoral lymphocytes T (LT).

In a particular embodiment, the method according to the invention is suitable to predict whether a subject suffers or is susceptible to suffer from a cancer and/or metastatic cancer.

As used herein, the term “CD70” is a member of the tumor necrosis factor superfamily (TNFSF), is typically present on activated memory B and T lymphocytes, natural killer (NK) cells and mature DCs. CD70 expression is necessary for an effective immune response, such as T cell activation and proliferation and memory cell generation, B cell activation and differentiation.

The human CD70 variant 1 has the following nucleotide sequence in the art SEQ ID NO:1:

| | |
|---|---|
| 1 | agagaggggc aggctggtcc cctgacaggt tgaagcaagt agacgcccag gagccccggg |
| 61 | agggggctgc agtttccttc cttccttctc ggcagcgctc cgcgccccca tcgcccctcc |
| 121 | tgcgctagcg gaggtgatcg ccgcggcgat gccggaggag ggttcgggct gctcggtgcg |
| 181 | gcgcaggccc tatgggtgcg tcctgcgggc tgctttggtc ccattggtcg cgggcttggt |
| 241 | gatctgcctc gtggtgtgca tccagcgctt cgcacaggct cagcagcagc tgccgctcga |
| 301 | gtcacttggg tgggacgtag ctgagctgca gctgaatcac acaggacctc agcaggaccc |
| 361 | caggctatac tggcaggggg gcccagcact gggccgctcc ttcctgcatg gaccagagct |
| 421 | ggacaagggg cagctacgta tccatcgtga tggcatctac atggtacaca tccaggtgac |
| 481 | gctggccatc tgctcctcca cgacggcctc caggcaccac cccaccaccc tggccgtggg |
| 541 | aatctgctct cccgcctccc gtagcatcag cctgctgcgt ctcagcttcc accaaggttg |
| 601 | taccattgcc tcccagcgcc tgacgcccct ggcccgaggg gacacactct gcaccaacct |
| 661 | cactgggaca cttttgcctt cccgaaacac tgatgagacc ttctttggag tgcagtgggt |
| 721 | gcgcccctga ccactgctgc tgattagggt tttttaaatt ttattttatt ttatttaagt |
| 781 | tcaagagaaa aagtgtacac acaggggcca cccggggttg gggtgggagt gtggtggggg |
| 841 | gtagtggtgg caggacaaga gaaggcattg agctttttct ttcattttcc tattaaaaaa |
| 901 | tacaaaaatc a |

The human CD70 variant 2 has the following nucleotide sequence in the art SEQ ID NO:2:

| | |
|---|---|
| 1 | agagaggggc aggctggtcc cctgacaggt tgaagcaagt agacgcccag gagccccggg |
| 61 | agggggctgc agtttccttc cttccttctc ggcagcgctc cgcgccccca tcgcccctcc |
| 121 | tgcgctagcg gaggtgatcg ccgcggcgat gccggaggag ggttcgggct gctcggtgcg |
| 181 | gcgcaggccc tatgggtgcg tcctgcgggc tgctttggtc ccattggtcg cgggcttggt |
| 241 | gatctgcctc gtggtgtgca tccagcgctt cgcacaggct cagcagcagc tgccgctcga |
| 301 | gtcacttggg tgggacgtag ctgagctgca gctgaatcac acaggacctc agcaggaccc |
| 361 | caggctatac tggcaggggg gcccagcact gggccgctcc ttcctgcatg gaccagagct |
| 421 | ggacaagggg cagctacgta tccatcgtga tggcatctac atggtacaca tccaggtgac |
| 481 | gctggccatc tgctcctcca cgacggcctc caggcaccac cccaccaccc tggccgtggg |
| 541 | aatctgctct cccgcctccc gtagcatcag cctgctgcgt ctcagcttcc accaagggct |
| 601 | ttttggattt tggaactggg gactcaaagt caagtgcttc ttacggcatt taatatggac |
| 661 | tgcacactgt tttatcccat taactcagct cgtgttcatg caagccctac aaagctggag |
| 721 | gaatcatcat tgttcccatt tcacagatga ggaaaacaga ggcgtaaacc gttgagtcat |
| 781 | ctgtccaaag ttactggttt tgtaacagct ggaacctcag tatttaacag ttgaggaaaa |
| 841 | tagtggcaga aaacctgagt cacaagcccg aggcaattca tctacatagt agtggactca |
| 901 | ggattccaag ccagataatc ccgcatcttg catccatgat ctcacttcta gaaggggaag |
| 961 | tgagaggagt ggtggggtgt cccagctgag ggagggaaaa tgagacccat gcgccacccg |
| 1021 | ccctcacaaa tctagccctg cagcccctct ccaaccttac ctggactcag aaccatgcat |
| 1081 | ctggaaaagc cacagacact cgacgccagc ctgtgaaagc agccgggagg gaggctgtat |
| 1141 | cctgcaaagc cacagggaag gggctgccca agaccatggg aacccaccac ttacatcagc |
| 1201 | gtgacctgga tgtgagacac agagtcaaag gagattatct ggcagcttta agattggact |
| 1261 | gcctgactat tcacaatagc aaagacttgg aaccaaccca aatgtccatc aatgatagac |
| 1321 | tggattaaga aaatgtggca catatacacc atggaatact atgcagccat aaaaacggat |
| 1381 | gagttcatgt ccttcatagg gacatggatg aagctggaaa ccatcattct gagcaaacta |
| 1441 | ttgcaaggac agaaaaccaa acaccgcatg ttctcactca taggtggaaa ttgaacaatg |
| 1501 | agaatacttg gacacaggga ggagaacatc acacaccggg gcctgtcgtg gagtgtggag |
| 1561 | tcgggggagg gggggaggga tagcattagg agaaatacct aatgtaaatg acaaattaat |
| 1621 | gggtgcagta aaccaacacg gcacatatat acatatgtaa caaatctgca cgttgtgcac |
| 1681 | atgtacccta gaacttaaag tataattaaa aaaaaaaaaa aaaaaagatt tgactgcctg |
| 1741 | gccaggcaca gtggctcatg cctataatcc taacactttg ggaggccgag gcgagcagat |
| 1801 | tgcctgagct gaagagttcg agaccagcct gggcaatatg gtgaaacccc gtttctacta |
| 1861 | aaatacaaaa aaaaaaaaat tagccaggcg tggcagcgtg cgcctgtagt ccccgcgact |
| 1921 | tggaggctga tgcaggagaa tcgctttaac ccgggaggca gaggttgcag tgagccgaga |
| 1981 | tcgagccact gtactccaga ctgggtgaca gagcgacact ctgtctcaaa aaaaaaaaaa |
| 2041 | agatttaact gccccactgg attttggaat tgcaggggac cttcagcccc ttcgtttgtc |
| 2101 | cagtttctcc catttggaat gggtatatcc aacgcctgta ccccattgta tataggaagt |
| 2161 | aactaacttg cttttgattt tacaggctca cagaagggac gtgccttgtc tcagatgaga |
| 2221 | ttttggactg tggacttttg agttaatgct gaaatgagtt aagactttgg gggatagtta |
| 2281 | ggaaggcatg actagtttga ggacattata ctcaatgaaa taagccagtc aaaaaggaaa |
| 2341 | aatatgtatt gtataaacta acttacatta atctaccaca taggattaaa actgttcaca |
| 2401 | ggaatataaa ataaaatttt ttaaata |

The human CD70 variant 1 has the following amino acid sequence in the art SEQ ID NO:3:

| | |
|---|---|
| 1 | mpeegsgcsv rrrpygcvlr aalvplvagl viclvvciqr faqaqqqlpl eslgwdvael |
| 61 | qlnhtgpqqd prlywqggpa lgrsflhgpe ldkgqlrihr dgiymvhiqv tlaicsstta |
| 121 | srhhpttlav gicspasrsi sllrlsfhqg ctiasqrltp largdtlctn ltgtllpsrn |
| 181 | tdetffgvqw vrp |

The human CD70 variant 2 has the following amino acid sequence in the art SEQ ID NO:4:

| | |
|---|---|
| 1 | mpeegsgcsv rrrpygcvlr aalvplvagl viclvvciqr faqaqqqlpl eslgwdvael |
| 61 | qlnhtgpqqd prlywqggpa lgrsflhgpe ldkgqlrihr dgiymvhiqv tlaicsstta |
| 121 | srhhpttlav gicspasrsi sllrlsfhqg lfgfwnwglk vkcflrhliw tahcfipltq |
| 181 | lvfmqalqsw rnhhcshftd eenrgvnr |

As used herein, the term “CD27” is a member of the tumor necrosis factor receptor superfamily. It is currently of interest to immunologists as a co-stimulatory immune checkpoint molecule. CD27 binds to ligand CD70, and plays a key role in regulating B-cell activation and immunoglobulin synthesis. A soluble form of CD27 (sCD27), a 32-kD protein identical to the extracellular domain of membrane-bound CD27, can be released after lymphocyte activation by differential splicing of the receptor protein or shedding from the cell surface by proteases.

The human CD27 has the following nucleotide sequence in the art SEQ ID NO:5:

| | |
|---|---|
| 1 | cttcaaaggt tggcttgcca cctgaagcag ccactgccca gggggtgcaa agaagagaca |
| 61 | gcagcgccca gcttggaggt gctaactcca gaggccagca tcagcaactg ggcacagaaa |
| 121 | ggagccgcct gggcagggac catggcacgg ccacatccct ggtggctgtg cgttctgggg |
| 181 | accctggtgg ggctctcagc tactccagcc cccaagagct gcccagagag gcactactgg |
| 241 | gctcagggaa agctgtgctg ccagatgtgt gagccaggaa cattcctcgt gaaggactgt |
| 301 | gaccagcata gaaaggctgc tcagtgtgat ccttgcatac cgggggtctc cttctctcct |
| 361 | gaccaccaca cccggcccca ctgtgagagc tgtcggcact gtaactctgg tcttctcgtt |
| 421 | cgcaactgca ccatcactgc caatgctgag tgtgcctgtc gcaatggctg gcagtgcagg |
| 481 | gacaaggagt gcaccgagtg tgatcctctt ccaaaccctt cgctgaccgc tcggtcgtct |
| 541 | caggccctga gcccacaccc tcagcccacc cacttacctt atgtcagtga gatgctggag |
| 601 | gccaggacag ctgggcacat gcagactctg gctgacttca ggcagctgcc tgcccggact |
| 661 | ctctctaccc actggccacc ccaaagatcc ctgtgcagct ccgattttat tcgcatcctt |
| 721 | gtgatcttct ctggaatgtt ccttgttttc accctggccg gggccctgtt cctccatcaa |
| 781 | cgaaggaaat atagatcaaa caaaggagaa agtcctgtgg agcctgcaga gccttgtcat |
| 841 | tacagctgcc ccagggagga ggagggcagc accatcccca tccaggagga ttaccgaaaa |
| 901 | ccggagcctg cctgctcccc ctgagccagc acctgcggga gctgcactac agccctggcc |
| 961 | tccaccccca ccccgccgac catccaaggg agagtgagac ctggcagcca caactgcagt |
| 1021 | cccatcctct tgtcagggcc ctttcctgtg tacacgtgac agagtgcctt ttcgagactg |
| 1081 | gcagggacga ggacaaatat ggatgaggtg gagagtggga agcaggagcc cagccagctg |
| 1141 | cgcctgcgct gcaggagggc gggggctctg gttgtaaaac acacttcctg ctgcgaaaga |
| 1201 | cccacatgct acaagacggg caaaataaag tgacagatga ccacc |

The human CD27 has the following amino acid sequence in the art SEQ ID NO:6:

| | |
|---|---|
| 1 | marphpwwlc vlgtlvglsa tpapkscper hywaqgklcc qmcepgtflv kdcdqhrkaa |
| 61 | qcdpcipgvs fspdhhtrph cescrhcnsg llvrnctita naecacrngw qcrdkectec |
| 121 | dplpnpslta rssqalsphp qpthlpyvse mleartaghm qtladfrqlp artlsthwpp |
| 181 | qrslcssdfi rilvifsgmf lvftlagalf lhqrrkyrsn kgespvepae pchyscpree |
| 241 | egstipiqed yrkpepacsp |

As used herein, the term “interaction between CD27 and CD70” refers to the interaction between the CD27 and its ligand CD70. Such interaction triggers a series of additional co-stimulatory signals, which results in the expansion and differentiation of memory and effector T cells. Despite the importance of CD27−CD70 interaction in the initiation of immune response, this continuous interaction may cause immune dysregulation and immunopathology. Studies elucidating CD27−CD70 interaction in a solid tumor is rare.

As used herein, the term “biological sample” refers to any sample obtained from a subject, such as a serum sample, a plasma sample, a urine sample, a blood sample, a lymph sample, tumor sample or a tissue biopsy. In a particular embodiment, biological sample for the determination of an expression level includes samples such as a blood sample, a lymph sample, or a biopsy.

In a particular embodiment, the biological sample is a blood sample. In another embodiment, the biological sample is a plasma sample. Typically, cancer patients and healthy donors were used to analyze sCD27 concentration by CD27 (Soluble) Human Instant ELISA Kit (ThermoFisher Scientific, Massachusetts, United States) according to the manufacturer's instructions. Data were acquired with MRX Revelation Microplate Reader (DYNEX Technologies, Virginia, United States).

In a further embodiment, the biological sample is tumor sample.

Typically, flow cytometry is performed with tumor sample as described in the Example. In a particular embodiment, the interaction between CD27 and CD70 is analyzed by immunofluorescent analysis. More particularly, the interaction Multiplex Immunofluorescence (mIF) Multiplex stained slides are imaged using the Vectra® Polaris™ Automated Quantitative Pathology Imaging system version 2 (Akoya). Using multispectral images obtained from single stained slides for each marker, a spectral library containing fluorophores emitting spectral peaks was created with inForm (version 2.4.6) image analysis software (PerkinElmer). Such software analysis allows the detection and segmentation of specific tissues through powerful pattern recognition algorithms, a machine-learning algorithm is trained to segment tumor from stroma and identify cells labelled.

As used herein, the term “level of soluble CD27” refers to the concentration of soluble CD27. Typically, the level or concentration of the soluble CD27 gene may be determined by any technology known by a person skilled in the art. In particular, the concentration may be measured at the genomic and/or nucleic and/or protein level. In a further embodiment, the soluble CD27 is characterized by Luminex, electrochemoluminescence or ultra-sensitive immunoassay (Simoa . . . ). Typically, the apoptosis, exhaustion, proliferation and cytotoxicity of lymphocytes T are measured by Flow cytometry by Single-cell RNA-seq, by in situ multiplex immunofluorescence and/or immunohistochemistry. In another embodiment, the production of cytokines and/or chemokines by lymphocytes T is measured by Luminex.

In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50 formamide, 5× or 6× SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the amount of sCD27 present in the plasma sample is detected by mass spectrometry.

In some embodiments, a score which is a composite of the level of the soluble CD27 is determined and compared to a reference value. When a high concentration of soluble CD27 than the reference value is determined, it is indicative that there is an interaction between CD27 and CD70. When a low concentration of soluble CD27 than the reference value is determined, it is indicative that there is not any interaction between CD27 and CD70. In a particular embodiment, the predetermined reference value is 48.2 UI/mL. In a further embodiment, the standard deviation value is 11.46±2. Said predetermined reference value is determined in healthy subjects. Typically, the predetermined reference value is a threshold value or a cut-off value, which can be determined experimentally, empirically, or theoretically. A threshold value can also be arbitrarily selected based upon the existing experimental and/or clinical conditions, as would be recognized by a person of ordinary skilled in the art. For example, retrospective measurement of the level of the soluble CD27 in properly banked historical plasma samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the soluble CD27 in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER. SAS, CREATE-ROC. SAS, GB STAT VI0.0 (Dynamic Microsystems, Inc. Silver Spring, Md., USA), etc.

In a particular embodiment, the method according to the invention further comprises a step of classification of subject by an algorithm and determining the interaction between CD27 and CD70.

Typically, the method of the present invention comprises a) quantifying the level of the soluble CD27 in the biological sample; b) implementing a classification algorithm on data comprising the quantified of sCD27 levels so as to obtain an algorithm output; c) determining the probability the interaction of CD27 and CD70 from the algorithm output of step b).

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF) selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In the context of the invention, inventors have shown that CD70 and CD27 interaction in ccRCC induce TILs apoptosis, leading to elevated sCD27 in peripheral blood in patients. When a low concentration of soluble CD27 than the reference value is determined, it is indicative that there is not any interaction between CD27 and CD70. It means that the series of additional and persistent co-stimulatory signals do not occur avoiding the risk of T cell dysfunction

Accordingly, the method according to the invention is suitable to predict the dysfunction of intratumoral lymphocytes T (LT).

Typically, the invention relates to a method for predicts the dysfunction of intratumoral lymphocytes (LT), comprising the following steps: i) determining the interaction between CD27 and CD70 according to the method as described above and ii) concluding that there is a dysfunction of intratumoral lymphocytes T when there is an interaction between CD27 and CD70 or concluding that there is not any dysfunction of intratumoral lymphocytes T when there is not any interaction between CD27 and CD70.

Typically, the invention relates to a method for predicts the dysfunction of intratumoral lymphocytes (LT), comprising the following steps: i): determining the level of soluble CD27 (sCD27) in a biological sample; ii) comparing the level of sCD27 quantified at step i) with its corresponding predetermined reference value and iii) concluding that there is a dysfunction of intratumoral lymphocytes (LT) when the level of sCD27 is higher than its corresponding predetermined reference value or concluding that there is not any dysfunction of intratumoral lymphocytes (LT) when he level of sCD27 is lower than its corresponding predetermined reference value.

As used herein, the term “intratumoral lymphocytes T” also called as tumor-infiltrating lymphocytes (TILs) refers to white blood cells that have left the bloodstream and migrated into a tumor. They are mononuclear immune cells, and can include a mixture of different types of cells (e.g., T cells, B cells, NK cells, macrophages) in variable proportions, with T cells usually being the most abundant. In a particular embodiment, in the context of the invention a TIL is a lymphocyte T cell.

As used herein the term “dysfunction of intratumoral lymphocytes T” refers to lymphocytes T cells which cannot be expanded and differentiated. Such lymphocytes are exhausted within the tumor and thus they cannot defend against tumor cells.

In a particular embodiment, the method according to the invention is suitable to predict whether a subject suffers or is susceptible to suffer from a cancer and/or metastatic cancer expressing CD70.

Accordingly, the invention relates to a method for predicting whether a subject suffers or is susceptible to suffer from a cancer and/or metastatic cancer expressing CD70 comprising the steps of: i) determining the interaction between CD27 and CD70 as described above; ii) and concluding that the subject suffers or is suffering from a cancer when there is an interaction between CD27 and CD70 or concluding that the subject does not suffer or is not suffering from a cancer when there is not any interaction between CD27 and CD70.

Typically, the invention relates to a method for predicting whether a subject suffers or is susceptible to suffer from a cancer and/or metastatic cancer expressing CD70 comprising the steps of: i) determining the level of soluble CD27 (sCD27) in a biological sample; ii) comparing the level of sCD27 quantified at step i) with its corresponding predetermined reference value and iii) concluding that the subject suffers or is suffering from a cancer and/or metastatic cancer expressing CD70 when the level of sCD27 is higher than its corresponding predetermined reference value or concluding that the subject does not suffer or is not suffering from a cancer and/or metastatic cancer expressing CD70 when the level of sCD27 is lower than its corresponding predetermined reference value.

In another embodiment, the invention is suitable to a method for predicting whether a subject suffering from a cancer will achieve a response to an immunotherapy treatment.

Typically, the invention relates to a method for predicting whether a subject suffering from a cancer and/or metastatic cancer expressing CD70 will achieve a response to an immunotherapy treatment comprising the steps of: i) determining the interaction between CD27 and CD70 as described above; ii) and concluding that the subject will achieve a response to an immunotherapy treatment when there is not any interaction between CD27 and CD70 or concluding that the subject will not achieve a response to an immunotherapy treatment when there is an interaction between CD27 and CD70.

In a further embodiment, the invention a method for predicting whether a subject suffering from a cancer and/or metastatic cancer expressing CD70 will achieve a response to an immunotherapy treatment comprising the steps of: i)quantifying the level of soluble CD27 in a biological sample; ii) comparing the level of soluble CD27 quantified at step i) with its corresponding predetermined reference values and iii) concluding that the subject will not respond to an immunotherapy treatment when the level of soluble CD27 is higher than its corresponding predetermined reference value or concluding that the subject will respond to an immunotherapy treatment when the level of soluble CD27 is lower than its corresponding predetermined reference value.

As used herein, the term “predicting” means that the subject to be analyzed by the method of the invention is allocated either into the group of subjects who suffers or is susceptible to suffer from a cancer and/or metastatic cancer, or into a group of subjects who does not suffer or is not suffering from.

As used herein, the term “cancer” refers to a malignant growth or tumor resulting from an uncontrolled division of cells. The term “cancer” includes primary tumors and metastatic tumors. In a particular embodiment, the cancer is a cancer expressing CD70. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the cancer is kidney cancer. As used herein, the terms “kidney cancer,” “renal cancer,” or “renal cell carcinoma” refer to cancer that has arisen from the kidney. The terms “renal cell cancer” or “renal cell carcinoma” (RCC), as used herein, refer to cancer which originates in the lining of the proximal convoluted tubule. More specifically, RCC encompasses several relatively common histologic subtypes: clear cell renal cell carcinoma, papillary (chromophil), chromophobe, collecting duct carcinoma, and medullary carcinoma. Clear cell renal cell carcinoma (ccRCC) is the most common subtype of RCC. In a particular embodiment, the cancer is a metastatic renal cell carcinoma.

In another embodiment, the cancer is lung cancer. As used herein, the term “lung cancer” includes, but is not limited to all types of lung cancers at all stages of progression like lung carcinomas metastatic lung cancer, non-small cell lung carcinomas (NSCLC) such as lung adenocarcinoma, squamous cell carcinoma, or small cell lung carcinomas (SCLC). In some embodiments, the subject suffers from a non-small cell lung carcinomas (NSCLC).

As used herein, the term “metastatic melanoma” refers to a cancer which does not respond to a classical treatment. The cancer may be resistant at the beginning of treatment or it may become resistant during treatment. The resistance to drug leads to rapid progression of metastasis. The resistance of cancer for the medication is caused by mutations in different genes, which are involved in the proliferation, divisions or differentiation of cells.

As used herein, the term “classical treatment” refers to any compound, natural or synthetic, used for the treatment of cancer and/or metastatic cancer. In a particular embodiment, the classical treatment refers to radiation therapy, immunotherapy, antibody therapy or chemotherapy.

As used herein, the term “subject” denotes a mammal, such as a rodent, a feline, a canine, and a primate. Particularly, the subject according to the invention is a human. More particularly, the subject according to the invention has or is susceptible to have a cancer expressing CD70. In a particular embodiment, the subject according to the invention has or is susceptible to have a cancer as described above. In another embodiment, the subject according to the invention has or is susceptible to have metastatic cancer.

In another embodiment, the subject according to the invention has or is susceptible to have renal cell carcinoma. In another embodiment, the subject according to the invention has or is susceptible to have clear cell renal cell carcinoma (ccRCC). In another embodiment, the subject according to the invention has or is susceptible to have lung cancer. In another embodiment, the subject according to the invention has or is susceptible to have NSCLC or SCLC. In another embodiment, the subject according to the invention has or is susceptible to have melanoma.

### Method for Treating a Cancer

In a second aspect, the invention relates to a method for treating cancer and/or metastatic cancer in a subject in need thereof comprising a step of administering said subject with a therapeutically effective amount of an inhibitor of interaction of CD27/CD70.

In a particular embodiment, the invention relates to a method for treating a cancer or a metastatic cancer in a subject identified as not having any interaction between CD27 and CD70 according to the method of the invention.

In a particular embodiment, the method according to the invention, wherein said cancer and/or metastatic cancer is a cancer expressing CD70.

Accordingly, the invention relates to a methods for treating a cancer or a metastatic cancer in a subject in need thereof comprising the following steps: i) determining the interaction between CD27 and CD70 according to the method as described above; ii) treating said subject with a therapeutically effective amount of an inhibitor of interaction of CD27/CD70 when there is not having any interaction between CD27 and CD70.

Typically, the invention relates to a methods for treating a cancer or a metastatic cancer in a subject in need thereof comprising the following steps: i) determining the level of soluble

CD27 (sCD27) in a biological sample; ii) comparing the level of sCD27 quantified at step i) with its corresponding predetermined reference value; iii) concluding that there is not any interaction between CD27 and CD70, when the level of sCD27 is lower than its corresponding predetermined reference value; and iv) treating said subject with a therapeutically effective amount of an inhibitor of interaction of CD27/CD70.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” denotes a mammal, such as a rodent, a feline, a canine, and a primate. Particularly, the subject according to the invention is a human. More particularly, the subject according to the invention has or is susceptible to have a cancer. In another embodiment, the subject according to the invention has or is susceptible to have metastatic cancer. In a particular embodiment, the subject according to the invention has or is susceptible to have a cancer expressing CD70. In a particular embodiment, the subject according to the invention has or is susceptible to have renal cell cancer (RCC). In a particular embodiment, the subject according to the invention has or is susceptible to have clear cell renal cell carcinoma (ccRCC). In particular embodiment, the subject has or is susceptible to have lung cancer. In particular embodiment, the subject has or is susceptible to have melanoma cancer.

As used herein, the term “cancer” refers to a malignant growth or tumor resulting from an uncontrolled division of cells as defined above. As used herein, the term “metastatic melanoma” refers to a cancer which does not respond to a classical treatment as described above. In a particular embodiment, the cancer is a cancer expressing CD70. The cancer according to the invention is described above.

As used herein, the term “inhibitor of interaction of CD27/CD70” also called as “antagonist” refers to a natural or synthetic compound that has a biological effect to inhibit the interaction of CD27 and CD70. Such inhibitor is an antagonist of CD27 or CD70. Such inhibition blocks the interaction between CD27 and CD70.

In a particular embodiment, the inhibitor of CD27/CD70 interaction is a peptide, peptidomimetic, polypeptide, decoy, small organic molecule, antibody, aptamers, siRNA or antisense oligonucleotide.

In a particular embodiment, the inhibitor of CD27/CD70 interaction is an inhibitor of CD27.

In a particular embodiment, the inhibitor of CD27/CD70 interaction is an inhibitor of CD70.

In a particular embodiment, the inhibitor of CD27/CD70 interaction is a polypeptide. The term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups).

In a particular embodiment, the polypeptide is a decoy peptide, or peptidomimetic that is capable of binding to CD27 or CD70.

In a particular embodiment, the inhibitor of CD27/CD70 interaction is petptidomimetic. The term “peptidomimetic” refers to a small protein-like chain designed to mimic a peptide.

In a particular embodiment, the peptidomimetic is a small protein-like chain designed to mimic a peptide such as CD27 or CD70 in the context of the invention. The term “peptidomimetic” or PM as used herein means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety includes non-amino acid chemical moieties. A peptidomimetic chemical moiety may also include one or more amino acid that are separated by one or more non-amino acid chemical units. A peptidomimetic chemical moiety does not contain in any portion of its chemical structure two or more adjacent amino acids that are linked by peptide bonds. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

In a particular embodiment, the peptidomimetic is a functional equivalent fragment of CD27 or CD70.

As used herein, a “functional equivalent” also known as a decoy, as “sink” or “trap” is a compound which is capable of binding to CD27 or CD70, thereby preventing their interaction together. Such peptidomimectic of CD27 or CD70 is in inactivated form.

As used herein, a “functional equivalent” also known as a decoy or “decoy receptor”, as “sink” or “trap” is a compound which is capable of binding to CD27 or CD70 thereby preventing their interaction together. More particularly, it is a compound that binds to a ligand, but is structurally incapable of signalling or presenting the agonist to signalling receptor complexes. A decoy acts as a molecular trap for the ligand, thereby preventing it from binding to its functional receptor. A decoy can be a CD27 or CD70 peptidomimetic or a fragment thereof. The term “functionally equivalent fragment” thus includes any equivalent of CD27 or CD70 obtained by altering the amino acid sequence, for example by one or more amino acid deletions, substitutions or additions such that the protein analogue retains the ability to bind to soluble CD70. Amino acid substitutions may be made, for example, by point mutation of the DNA encoding the amino acid sequence. Functional equivalents include molecules that bind CD27 or CD70.

The term “variant” is, with respect to peptidomimetics, to be understood as a peptidomimetic which differs in comparison to the peptidomimetic from which it is derived by one or more changes in the amino acid sequence. The peptidomimetic from which a protein variant is derived is also known as the parent polypeptide. Typically, a variant is constructed artificially, preferably by gene-technological means. Typically, the parent polypeptide is a wild-type protein or wild-type protein domain. The variants usable in the present invention may also be derived from homologs, orthologs, or paralogs of the parent polypeptide. The changes in the amino acid sequence may be amino acid exchanges, insertions, deletions, N-terminal truncations, or C-terminal truncations, or any combination of these changes, which may occur at one or several sites. In a particular embodiment, a variant usable in the invention exhibits a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid exchanges may be conservative and/or non-conservative. In preferred embodiments, a variant usable in the present invention differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid exchanges, preferably conservative amino acid changes. Alternatively or additionally, a “variant” as used herein can be characterized by a certain degree of sequence identity to the parent polypeptide from which it is derived. More precisely, a protein variant in the context of the invention exhibits at least 80% sequence identity to its parent polypeptide. Particularly, the sequence identity of protein variants is over a continuous stretch of 20, 30, 40, 45, 50, 60, 70, 80, 90, 1 00, 150, 200, 250, 300, 400, 500, 600 or more amino acids, more preferably over the entire length of the reference polypeptide (the parent polypeptide). The term “at least 80% sequence identity” is used throughout the specification with regard to polypeptide sequence comparisons. This expression particularly refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide. Particularly, the polypeptide in question and the reference polypeptide exhibit the indicated sequence identity over a continuous stretch as specified above.

The sequence identity of a “variant” peptidomimetic of the invention can be determined over an identified range of an amino acid sequence of CD27 or CD70 with reference to the entire amino acid sequence of an identified CD27 or CD70 decoy peptidomimetic. When determining the percent sequence identity for a variant peptidomimetic, the sequence alignment may omit any specifically excluded amino acid residues. For example, for a variant peptidomimetic of a CD27 or CD70 decoy peptidomimetic that exhibits at least 80% sequence identity to amino acids of SEQ ID NO: 3; SEQ ID NO: 4 or SEQ ID NO: 6, the sequence alignment for comparison may occur across only amino acids or may take into account two or more identified ranges of amino acid sequences within the full length CD27 or CD70 decoy peptidomimetic.

In a particular embodiment, the inhibitor of CD27/CD70 interaction is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of CD27/CD70 interaction is a small organic molecule. The term “small organic molecule” refers to a molecule of a size comparable to those organic molecules generally used in pharmaceuticals. The term excludes biological macro molecules (e.g., proteins, nucleic acids, etc.). Preferred small organic molecules range in size up to about 5000 Da, more preferably up to 2000 Da, and most preferably up to about 1000 Da.

In a particular embodiment, the inhibitor of CD27/CD70 interaction is an antibody. Such antibody blocks the interaction CD27/CD70. In a particular embodiment, the antibody is neutralizing monoclonal antibody of CD70. In another embodiment, the antibody is neutralizing monoclonal antibody of CD27.

As used herein, the term “antibody” is used in the broadest sense and specifically covers monoclonal antibodies, polyclonal antibodies, multi specific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see (Kabat et al., 1991), specifically incorporated herein by reference). Diabodies, in particular, are further described in EP 404,097 and WO 93/11161; whereas linear antibodies are further described in (Zapata et al., 1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of (Beckman et al., 2007; Holliger & Hudson, 2005; Le Gall et al., 2004; Reff & Heard, 2001; Reiter et al., 1996; Young et al., 1995) further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor of CD27/CD70 interaction is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular embodiment, the inhibitor is an intrabody having specificity for CD27 or CD70. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In a particular embodiment, the inhibitor of CD27/CD70 interaction is an antibody anti-CD27.

In a particular embodiment, the inhibitor of CD27/CD70 interaction is an antibody anti-CD70.

In a particular embodiment, the inhibitor of CD27/CD70 interaction is an antibody anti-CD70 wherein said antibody anti-CD70 is selected in the following group consisting of but not limited to: HPAB-0095-LSX; Human Anti-CD70 Recombinant Antibody (clone 2H5); HPAB-0469-WJ-F(E); Human Anti-CD70 Recombinant Antibody (clone Ab2); HPAB-0095-LSX-F(E); Human Anti-CD70 Recombinant Antibody (clone 2H5); HPAB-0468-WJ-F(E); Human Anti-CD70 Recombinant Antibody (clone Ab1); HPAB-AP586-YC; Human Anti-CD70 Recombinant Antibody (clone h1F6-HHLA); HPAB-AP589-YC-F(E); Human Anti-CD70 Recombinant Antibody (clone h1F6-HMLA); TAB-336LC-F(E); Anti-Human CD70 Therapeutic Antibody Fab Fragment (Mb VLVH); or Rat Anti-Cd70 Recombinant Antibody (clone FR70).

In a particular embodiment, the inhibitor of CD27/CD70 interaction is ARGX-110. ARGX-110 also called as Cusatuzumab is developed by Argenx BVBA and has been described in WO2012/123586 and Aftimos et al 2017.

In a particular embodiment, the inhibitor of CD27/CD70 interaction is Vorsetuzumab mafodotin (SGN-75). SGN-75 is an antibody-drug conjugate (ADC), the humanized monoclonal antibody, vorsetuzumab, conjugated with noncleavable monomethyl auristatin F (MMAF), a cytotoxic agent.

As used herein the terms “administering” or “administration” refer to the act of injecting or otherwise physically delivering a substance as it exists outside the body (e.g. an inhibitor of CD27/CD70 interaction) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the inhibitor of CD27/CD70 interaction is administered orally.

In another aspect, the invention relates to an inhibitor of CD27/CD70 interaction and ii) a classical treatment, as a combined preparation for use in the prevention and/or treatment of cancer and/or metastatic cancer expressing CD70 in a subject in need thereof.

In a particular embodiment, the invention relates to i) an inhibitor of CD27/CD70 interaction and ii) a classical treatment for use by simultaneous, separate or sequential administration in the prevention and/or treatment of cancer and/or metastatic cancer expressing CD70 in a subject in need thereof.

As used herein, the terms “combined treatment”, “combined therapy” or “therapy combination” refer to a treatment that uses more than one medication.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein, the term “classical treatment” refers to treatments well known in the art and used to treat cancer. In the context of the invention, the classical treatment refers to targeted therapy, radiation therapy, immunotherapy or chemotherapy.

In a particular embodiment, the invention relates to i) inhibitor of CD27/CD70 interaction according to the invention and ii) a radiation therapy used as a combined preparation for use in the prevention and/or treatment of cancer and/or metastatic cancer expressing CD70 in a subject in need thereof.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates to i) an inhibitor of inhibitor of CD27/CD70 interaction according to the invention and ii) a chemotherapy used as a combined preparation for use in the prevention and/or treatment of cancer and/or metastatic cancer expressing CD70 in a subject in need thereof.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates to i) an inhibitor of CD27/CD70 interaction according to the invention and ii) an immune checkpoint inhibitor, as a combined preparation for use in the prevention and/or treatment of cancer and/or metastatic cancer expressing CD70 in a subject in need thereof.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+T cells from the naive to effector cell phenotype, however tumor-specific human CD8+T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to -N-(3-bromo-4-fluorophényl)-N′-hydroxy-4-{[2-(sulfamoylamino)-éthyl]amino}-1,2,5-oxadiazole-3 carboximidamide:

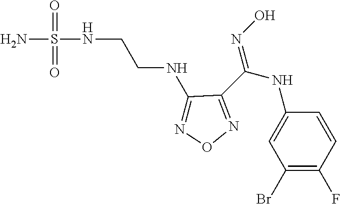 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

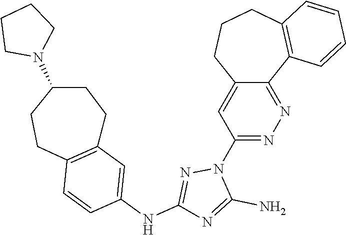 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In a particular embodiment, the invention relates to i) an inhibitor of CD27/CD70 interaction according to the invention and ii) anti-PD1 antibody, as a combined preparation for use in the prevention and/or treatment of cancer and/or metastatic cancer expressing CD70 in a subject in need thereof.

In a particular embodiment, the invention relates to i) an inhibitor of CD27/CD70 interaction according to the invention and ii) anti-PDL1 antibody, as a combined preparation for use in the prevention and/or treatment of cancer and/or metastatic cancer expressing CD70 in a subject in need thereof.

In a particular embodiment, the invention relates to i) an inhibitor of CD27/CD70 interaction according to the invention and ii) anti-PD2 antibody, as a combined preparation for use in the prevention and/or treatment of cancer and/or metastatic cancer expressing CD70 in a subject in need thereof.

In a particular embodiment, the invention relates to i) an inhibitor of CD27/CD70 interaction according to the invention and ii) anti-CTLA4 antibody, as a combined preparation for use in the prevention and/or treatment of cancer and/or metastatic cancer expressing CD70 in a subject in need thereof.

In a particular embodiment, the invention relates to i) an inhibitor of CD27/CD70 interaction according to the invention and ii) an anti-angiogenesis compound, as a combined preparation for use in the prevention and/or treatment of cancer and/or metastatic cancer expressing CD70 in a subject in need thereof.

As used herein, the term “angiogenesis” refers to a physiological process involving the growth of new blood vessels from preexisting vessels. Angiogenesis is a combinatorial process that is regulated by a balance between pro- and anti-angiogenic molecules. Angiogenic stimuli (e.g. hypoxia or inflammatory cytokines) result in the induced expression and release of angiogenic growth factors such as vascular endothelial growth factor (VEGF) or fibroblast growth factor (FGF).

As used herein, the term “anti-angiogenesis” refers to any molecule which can inhibit (anti-angiogenesis) the creation of new blood vessels. Typically, anti-angiogenesis compound is well known in the art and refers to the following compounds but not limited to bevacizumab (Avastin, anti-VEGF), itraconazole (anti-VGFR), carboxyamidotriazole, TNP-470 (an analog of fumagillin), CM101, IFN-α, IL-12, platelet factor-4, suramin, SU5416, thrombospondin, VEGFR antagonists, angiostatic steroids+heparin, Cartilage-Derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αVβ3 inhibitors, linomide, ramucirumab, tasquinimod, ranibizumab, sorafenib (Nexavar), sunitinib (Sutent), pazopanib (Votrient), everolimus (Afinitor), cabozantinib.

By a “therapeutically effective amount” is meant a sufficient amount of an inhibitor of CD27/CD70 interaction for use in a method for the treatment of cancer at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

The inhibitor of CD27/CD70 interaction alone or with a classical treatment as described above may be combined with pharmaceutically acceptable excipients, and optionally sustained-release matrices, such as biodegradable polymers, to form pharmaceutical compositions.

Accordingly, in another aspect, the invention relates to a pharmaceutical composition comprising inhibitor of CD27/CD70 interaction and pharmaceutically acceptable excipients.

In a particular embodiment, the pharmaceutical composition according to the invention comprising i) an inhibitor of CD27/CD70 interaction and ii) a classical treatment for use by simultaneous, separate or sequential administration in the prevention and/or treatment of cancer and/or metastatic cancer expressing CD70 in a subject in need thereof.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kits or Devices of the Present Invention

In another aspect, the present invention relates to a kit or device for performing the method of the present invention, comprising means for determining the level of the soluble CD27 in a biological sample.

In some embodiments, the kit or device comprises at least one binding partner (e.g. antibody or aptamer) specific for the soluble CD27 (immobilized or not on a solid support as described above). In some embodiments, the kit or device can include a second binding partner (e.g. antibody or aptamer) of the present invention which produces a detectable signal. Examples of kits include but are not limited to ELISA assay kits, and kits comprising test strips and dipsticks.

In some embodiments, the kit or device of the present invention further comprises a microprocessor to implement an algorithm on data comprising the level of soluble CD27 in the sample so as to determine the probability of responding to an immune checkpoint inhibitor. In some embodiments, the kit or device of the present invention further comprises a visual display and/or audible signal that indicates the probability determined by the microprocessor.

In some embodiments, the kit or device of the present invention comprises:
a mass spectrometer;
a receptacle into which the biological sample is placed, and which is connectable to the mass spectrometer so that the mass spectrometer can quantify the level of soluble CD27 in the sample;
a microprocessor to implement an algorithm on data comprising the levels of soluble CD27 in the sample so as to determine the probability of responding to an immune checkpoint inhibitor;
a visual display and/or audible signal that indicates the probability determined by the microprocessor.

### Method of Screening

A further object of the present invention relates to a method of screening a drug suitable for the treatment of cancer and/or metastatic cancer comprising i) providing a test compound and ii) determining the ability of said test compound to inhibit CD27/CD70 interaction.

Any biological assay well known in the art could be suitable for determining the ability of the test compound to inhibit the CD27/CD70 interaction. In some embodiments, the assay first comprises determining the ability of the test compound to bind to CD27 or CD70. In some embodiments, a population of cells is then contacted and activated so as to determine the ability of the test compound to inhibit the CD27/CD70 interaction. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting CD27/CD70 interaction, as determined using in vitro methods described herein, are likely to exhibit similar modulatory capacity in applications in vivo. Typically, the test compound is selected from the group consisting of peptides, petptidomimetics, small organic molecules, antibody, decoy, aptamers or nucleic acids. For example the test compound according to the invention may be selected from a library of compounds previously synthesised, or a library of compounds for which the structure is determined in a database, or from a library of compounds that have been synthesised de novo.

In some embodiments, the test compound may be selected form small organic molecules, decoy or antibody.

The invention will be further illustrated by the following figures and examples. However, these examples and figures should not be interpreted in any way as limiting the scope of the present invention.

### FIGURES

FIG. 1: Overexpression of CD27 and CD70 gene expression in ccRCC. CD27 (A) and CD70 (B) mRNA expression were compared in normal kidney tissues from non-cancer individuals (n=28), tumor tissues (n=530) and normal tissues adjacent to tumors (NAT) (n=72) from ccRCC patients. Data was presented as the mean with SD. Unpaired t test was used to determine the significance. (C) Correlation analysis (n=530) between CD27 and CD70 mRNA expression in ccRCC. Pearson correlation coefficient (r) and significance levels (P value) were shown. (D-F) Kaplan-Meier plot of the overall survival of ccRCC patients (n=376). Patients were divided into 2 groups (high and low) based on the median of CD27 (8.12) and CD70 (10.265) mRNA expression. “CD27 high CD70 high” group (n=124) referred to patients with high expression of CD27 and CD70 while “CD27 low and CD70 low” group (n=124) referred to patients with low expression of CD27 and CD70. Significance was determined using the log-rank Mantel-Cox test. Values of mRNA data were in log 2 scale for clarity. Values of P<0.05 were considered statistically significant. * P<0.05, **** P<0.0001.

FIG. 2: CD27−CD70 interaction in ccRCC demonstrated by multiplex IHC. Quantification of interacted CD27 and interacted CD70 in ccRCC (n=25). Data were presented with dots and mean with SD.

FIG. 3: CD27+T cells in tumors express cleaved caspase 3. Cleaved caspase 3 percentage in CD27+/−T cells (n=2).

FIG. 4: sCD27 in the plasma correlate with expression of intratumoral CD27 and the levels of interaction of CD27 with CD70. (A) Concentrations of plasma sCD27 from ccRCC patients (n=44) and healthy donors (n=15) were determined by ELISA. Significance was determined by unpaired t test. Data are presented with dots and mean with SD. sCD27 plotted against CD27+ cell numbers per field (B) and numbers of interacted CD27+ cells (CD27 within 30 μm of CD70) per field (C) in tumors (n=25). Pearson correlation coefficient (r) and significant levels (P value) are presented. Values of P<0.05 were considered statistically significant. ** P<0.01, *** P<0.001.

### EXAMPLE

### Material & Methods

### Patients and Samples

Two cohorts from prospective studies were enrolled at the Hôpital Européen Georges Pompidou (HEGP) (Paris, France). Patients from cohort Colcheckpoint (CPP Ile-de-France 2015 Aug. 4 MS2) were diagnosed with metastatic ccRCC and were treated by anti-PD-1/PD-L1. Patients from cohort ExhauCRF (CPP Ile-de-France 2016 Jul. 8) were diagnosed with localized ccRCC. For ELISA, 26 plasma samples from Colcheckpoint before anti-PD-1/PD-L1 treatment and 18 plasma samples from ExhauCRF at the time of diagnosis were collected. Plasma of 15 healthy donors from Etablissement Français du Sang were selected as control. For multiplex immunofluorescence (mIF), 7 formalin-fixed, paraffin-embedded (FFPE) tumor tissues from Colcheckpoint collected before immunotherapy and 18 from ExhauCRF cohort were selected. For flow cytometry, 2 fresh tumors were collected from patients confirmed with ccRCC on the day of surgery. Clinical characteristics, such as the histopathology, the performance status scale (ECOG), the TNM stage and the survival data for each patient were collected prospectively.

### Multiplex Immunofluorescence (mIF) Staining

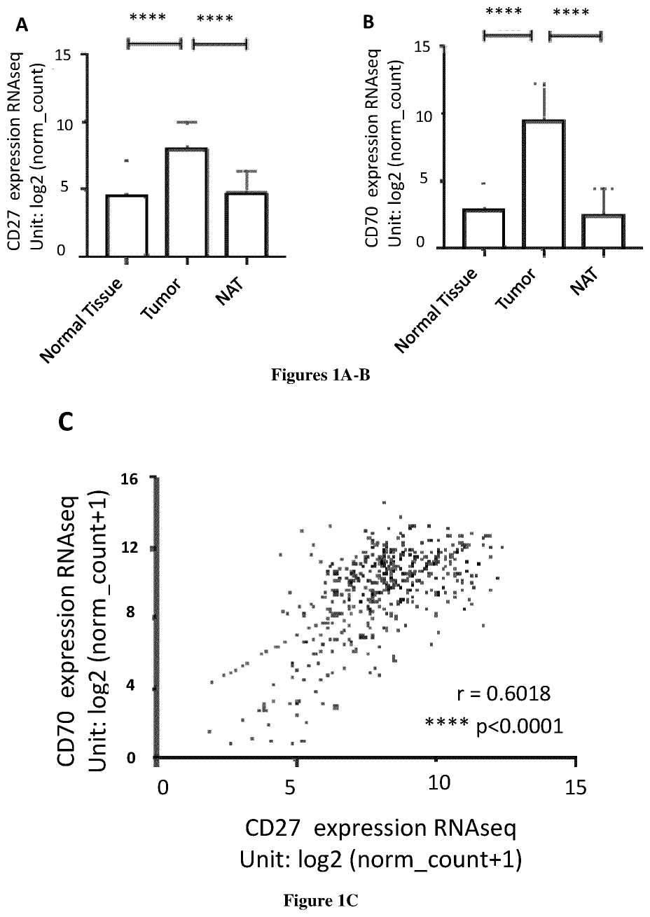

We developed 2 mIF panels, which were CD4 panel (CD4−CD27−CD70-PAX8) and CD8 panel (CD8−CD27−CD70-PAX8). Pax8 is a transcriptional factor with strong nuclear expression in most neoplastic cells in all histologic types of human primary or metastatic RCC. mIF panel composed of these different markers was developed manually and then applied to all FFPE tumor tissues automatically on the LEICA Bond RX with the same protocol. Slides from FFPE tissue were heated at 57° C. for 2 h before staining. Residual paraffin was removed in three successive Bioclear New dewaxing solution (Biognost, Zagreb, Croatia) for 3 minutes each. After the tissue rehydration using three serially diluted ethanol (100%, 75%, 50%) to distilled water for 2 minutes each, tissues were then fixed for 15 minutes in Formaldehyde fixation neutral buffer (NB) (Biognost) and washed with distilled water. Subsequently, antigen retrieval was performed using pH9 Target Retrieval (Agilent, California, United States) and microwave treatment for 45 seconds at 1000 watts followed by 30 minutes at 100 watts. Blocking was performed with blocking buffer devoid of animal-derived proteins (Cell Signaling Technology (CST), Massachusetts, United States) for 15 minutes, then slides were incubated with the primary antibody diluted in SignalStain® Antibody Diluent (CST) for 30 minutes. After washing in Tris-Buffered Saline, 0.1% Tween® 20 Detergent (TBST) (Agilent), incubation with secondary antibody coupled with horseradish peroxidase (HRP) (ImmunoReagents, North Carolina, United States) was performed for 15 minutes. The slides were washed in TBST and the CF® Dye Tyramide (Biotium, California, United States) was applied for 10 minutes. Slides were then microwaved to strip the primary and secondary antibodies, washed, and blocked again using blocking solution. The process was repeated until the fourth marker was labeled (FIG. 1). At the last step, staining with DAPI (PerkinElmer, Massachusetts, United States) was applied and then washed in distilled water. Slides were mounted using EverBrite Mounting Medium (Biotium). Finally, the slides were read on a Vectra Polaris fluorescence microscope (Akoya Biosciences, California, United States). Antibodies used in mIF panel are listed in Table 1.

TABLE 1
Antibody information of multiplex IHC panel

| [symbol] | | | | | |
|---|---|---|---|---|---|
| Position | Primary Ab | Concentration | Secondary Ab | TSA-Dye | [symbol] |
| 1 | CD70 | 1:500 | Anti-Mouse | 488A | 1:450 |
| | R&D | | [symbol] | [symbol] | |
| | MAB2738 | | GAMHRP-050 | 92171 | |
| 2 | CD4 | 1:500 | Anti-Rabbit | CF680R | 1:450 |
| | [symbol] | | [symbol] | [symbol] | |
| | AB133616 | | GARHRP-050 | 92196 | |
| 2 | CD8 | 1:500 | Anti-Mouse | CF680R | 1:450 |
| | CST | | [symbol] | [symbol] | |
| | 70306S | | GAMHRP-050 | 92196 | |
| 3 | CD27 | 1:12500 | Anti-Rabbit | 543 | 1:450 |
| | AB131254 | | [symbol] | [symbol] | |
| | | | GARHRP-050 | 92172 | |
| 4 | PAX8 | 1:1000 | Anti-Rabbit | 594 | 1:450 |
| | [symbol] | | [symbol] | [symbol] | |
| | AB191870 | | GARHRP-050 | 92174 | |

### Multispectral Imaging, Phenotyping and Spatial Analysis

Multiplex stained slides were imaged using the Vectra® Polaris™ Automated Quantitative Pathology Imaging system version 2 (Akoya). Regions of interest were selected and 10 representative images per patient were used for analysis. Using multispectral images obtained from single stained slides for each marker, a spectral library containing fluorophores emitting spectral peaks was created with inForm (version 2.4.6) image analysis software (PerkinElmer). This spectral library was then used to separate each multispectral image into its individual components, which allows for the color-based identification of all 5 markers in a single image using inForm software.

Selected images were exported as component data with inForm for phenotyping and spatial analysis in HALO software (Indica labs, New Mexico, United States). Highplex FL module in HALO was used for cellular phenotyping. Cells were segmented according to DAPI (nucleus) staining. Phenotyping was realized by setting an appropriate threshold to each marker according to the fluorescent intensity, and the same algorithm was applied to all images for homogeneity. To determine the cellular interaction, spatial analysis was realized based on cellular phenotypes with HALO. Cellular distance is calculated from the center of each cell. CD27 positive cells within 30 μm of CD70 positive cells were nominated as “interacted CD70 and center CD70 positive cells with interacted CD27 as “interacted CD70”. Interacted CD27 and interacted CD70 were calculated.

### Flow Cytometry on Fresh Tumors for the Characterization of TILs in ccRCC

Two fresh tumors of ccRCC patients were collected. For digestion, fresh tumor was first cut into pieces then incubated with 25 mL Hank's Balance Salt Solution (HBSS) with calcium and magnesium (Lonza, Basel, Switzerland), lmg/ml final concentration collagenase (Roche, Basel, Switzerland) and 15 mg/ml DNAase (Roche) at 37° C. for 1 hour. Tumor tissues were filtered and washed with 200 μl EDTA (Sigma-Aldrich, Missouri, United States) and HBSS at 1000 r for 10 minutes. After removal of supernatant, residual cells were resuspended with HBSS. Cell numbers were counted with Trypan Blue.

1 million cells per tube were used for staining. Cells were first stained with Zombie Nir (Biolegend, California, United States) for 30 minutes to distinguish living cells from dead cells. After washing with cell staining buffer (Biolegend), cells were then stained with the following monoclonal antibodies directly labeled with fluorophore (Table 2) for 30 minutes in dark. After the staining, cells were washed again and resuspended in 200 uL cell staining buffer. Samples were acquired in cytometer (Navios 10 colors, Beckman Coulter) and data were analyzed with software Kaluza (version 1.2).

TABLE 2
Antibody information for flow cytometry

| Antibody | Isotype | Company | Catalog |
|---|---|---|---|
| CD3 BV510 | Mouse IgG1 k | Biolegend | 300448 |
| CD4 BV421 | Mouse IgG1 k | Biolegned | 300532 |
| CD8 PE | Mouse IgG1 k | BD | 345773 |
| IgG1 APC | Mouse IgG1 k | Biolegend | 400122 |
| CD27 APC | Mouse IgG1 k | Biolegend | 302810 |
| Caspase 3 | — | Biotium | 10403 |
| substrate AF488 | | | |

### Determination of sCD27 in Plasma

10 uL plasma from ccRCC patients and healthy donors were used to analyze sCD27 concentration by CD27 (Soluble) Human Instant ELISA Kit (ThermoFisher Scientific, Massachusetts, United States) according to the manufacturer's instructions. Data were acquired with MRX Revelation Microplate Reader (DYNEX Technologies, Virginia, United States).

### Gene Expression Analysis from TCGA Database

Gene expression analysis was performed using UC Santa Cruz Cancer Genomics Browser (http://xena.ucsc.edu/). To compare CD27 and CD70 gene expression across 31 types of human solid tumors, TCGA PanCan Study was created using normalized gene-level RNA-seq data (n=9575) downloaded from The Cancer Genome Atlas (TCGA) and Pan-Cancer Atlas database. To compare CD27 and CD70 gene expression in tumor and normal tissue, normalized mRNA data from ccRCC tumor tissue (n=530), normal kidney tissue (n=28) and normal tissue adjacent to tumors (NAT) (n=72) were downloaded from the TCGA-PanCan Study and GTEX Study. Tumor tissues and NAT tissues were derived from ccRCC patients and normal kidney tissues from individuals who do not have cancers. Corresponding clinical data (n=376) such as overall survival was also downloaded for Kaplan-Meier survival analysis.

### Statistical Analysis

One-way Anova was performed with UCSC Xena tool for the comparison of CD27 and CD70 gene expression across different types of solid tumors. Other statistical analyses were performed using GraphPad Prism software version 8. Two-tailed unpaired t test was used to evaluate the quantifications between two groups as appropriate. For the correlation analyses, the Pearson's correlation coefficient (r) and P value was calculated. For survival analyses, Kaplan-Meier plots were depicted, and statistical differences were evaluated using the log-rank Mantel-Cox test. A P value <0.05 was considered statistically significant.

### Tissue Processing RNA Extraction, Single-cell RNA Sequencing

Fresh tumors were collected as described above. After tumor dissociation, cells were stained with a fixable viability stain FVS 520 (eBioscience), an anti-CD8a labeled with APC fluorophore (Biolegend) and an anti-NKP46 labeled with BV421 (Biolegend) in order to eliminate natural killer cells from the analysis during the cell sorting FACS-enriched T cells were loaded on a 10× Chromium (10× Genomics) and libraries were prepared using a Single Cell 5′ Reagent Kit (V1 chemistry, 10× Genomics) according to the manufacturer's protocol, targeting 3000 recovered cells per sample. Single cells were partitioned and barcoded into droplets together with gel beads coated with oligos harbouring unique barcodes, molecular identifiers (UMI), and template switch oligo (TSO) sequences, followed by in droplet reverse transcription to generate barcoded full-length cDNA. cDNA was subsequently recovered from droplets, then cleaned up with DynaBeads MyOne Silane Beads (Thermo Fisher Scientific), then amplified with the following protocol: 98° C.—45 s; 12× (98° C.—20 s, 67° C.—30 s, 72° C.—1 min), 72° C.—1 min; held at 4° C. Amplified cDNA product was cleaned up using the SPRI select Reagent Kit (Beckman Coulter). Gene expression libraries were constructed following these steps: (1) fragmentation, end repair and A-tailing; (2) size selection with SPRI select beads; (3) adaptor ligation; (4) post-ligation cleanup with SPRI select beads; (5) sample index PCR with the following protocol: 98° C.—45 s; 12× (98° C.—20 s, 54° C.—30 s, 72° C.—20 s), 72° C.—1 min; held at 4° C. and final cleanup with SPRI select beads. Libraries quality was assessed using a dsDNA High Sensitivity Assay Kit and Bioanalyzer Agilent 2100 System. Libraries were quantified with dsDNA HS kit and Qubit 2.0. Indexed libraries were pooled and sequenced on an Illumina HiSeqX using paired-end 150 bp as sequencing mode, targeting at least 50 000 reads per cell.

### scRNA-Seq Data Analysis

All scRNA-seq data were processed with the Cellranger pipeline (version 2.1.1). This step included demultiplexing of raw base call (BCL) files into FASTQ files, reads alignment on human genome assembly GRCh38-3.0.0 using STAR, and counting of unique molecular identifier (UMI). The Seurat (v3.1.1) workflow was used to read the Raw data in R (3.6.1). Raw counts of each donor were normalized with the Seurat SCTransform function using the default parameters and integrated in a single object using the Seurat CCA integration algorithm.As a quality-control step, we first filtered out low-quality cells: cells with less than 200 genes detected and more than 5,000 genes detected. Cells with more than 5,000 mitochondrial reads were removed as well as cells for which the ratio of reads over mitochondrial reads exceeded 6.5. Reads aligning to mitochondrial genes or ribosomal proteins were removed from the analysis. Following these quality-control criteria, 13,389 T CD8 (patient P1=2,006 cells; patient P2=7,002 cells; patient P3=1,654 cells; patient P4=2,727 cells) were finally conserved. For each patient, single cell were selected as “CD27 positive” (nUMI corresponding to CD27 gene>0) or “CD27 negative”. The differentially expressed genes between CD27 positive cells and CD27 negative cells were identified using Student t-test and Benjamini-Hochberg p-value. Genes were considered differentially expressed if p value corrected was ≤0.05 and log FC≥0.4). 412 genes were up-regulated in CD27 negative cells and 330 genes were up-regulated in CD27 positive cells. Morpheus tool (https://software.broadinstitute.org/morpheus/) was used for Heatmap visualization. scRNA-seq data sorted from renal cell carcinoma samples are available on NCBI's Gene Expression Omnibus (GEO) Archive platform (https://www.ncbi.nlm.nih.gov/geo/) under accession number GSE160243.

### Results

### Distinct Expression Pattern of CD70 and CD27 in ccRCC Correlates with Patient Survival

To validate the frequent expression of CD70 in ccRCC (Adam et al., 2006; Jilaveanu et al., 2012), CD70 expression at mRNA levels, as well as its ligand CD27, were compared across 31 types of human solid tumors from cohort TCGA and Pan-Can Atlas (n=9575). As result, CD70 is the most frequently expressed in kidney renal clear cell carcinoma (KIRC) (data not shown). Similarly, CD27 expression in KIRC is also at high prevalence compared to other solid tumors (data not shown). When we focused on ccRCC, we found that CD27 and CD70 expression were significantly higher in tumors than in normal kidney tissues and in normal tissues adjacent to tumor (NAT) (FIG. 1A, 1B). Besides, CD27 is significantly correlated with CD70 in tumor (FIG. 1C). To study the impact of this distinct expression pattern of CD27 and CD70 on clinical outcomes, overall survival analysis was performed using data from TCGA-PanCan study (n=530). Patients were divided into 2 groups (high and low) based on the median of mRNA data at log 2 scale. The median threshold is 8.12 for CD27 and 10.265 for CD70. CD27 or CD70 alone had no impact on patient overall survival (FIG. 1D, 1E). Given the correlation of CD27 and CD70 in tumors, we combined 2 markers and divided patients into 2 groups, which were “CD27 high CD70 high” (n=124) and “CD27 low CD70 low” (n=124) respectively. Interestingly, we observed a significantly poor OS in “CD27 high CD70 high” group (FIG. 1F), suggesting that a synergic role of CD27 and CD70 has a negative impact on ccRCC patient prognosis.

### Interaction of CD70⁺ Tumor Cells and CD27⁺T cells in ccRCC

To better understand the synergic role of CD27 and CD70 in ccRCC tumor microenvironment (TME), we performed 5-color mIF on FFPE tumor tissues from 25 patients. Two staining panels were designed: CD4 panel (CD4, red; CD27, yellow; CD70, green; PAX8, orange; DAPI (nuclear stain), blue) and CD8 panel (CD8, red; CD27, yellow; CD70, green; PAX8, orange; DAPI, white). PAX8 was used to identify tumor cells. Composite images with 5 markers after multispectral imaging and corresponding single stained images were presented (data not shown). After cell segmentation of the nucleus, cytoplasm and membrane and measurement of the fluorescent intensity of each compartment, cells were phenotyped and counted by HALO software (data not shown). Algorithm composed of appropriate intensity threshold of each marker was applied to all patients for homogeneity. Flow cytometry allows for accurate phenotyping of cells, but fails to capture the spatial relationships that can better reflect the cellular interaction. To examine this, after phenotyping, each cell was assigned coordinates that could be used to determine the intracellular distances. Cell numbers within certain distance of another cell population can be also calculated.

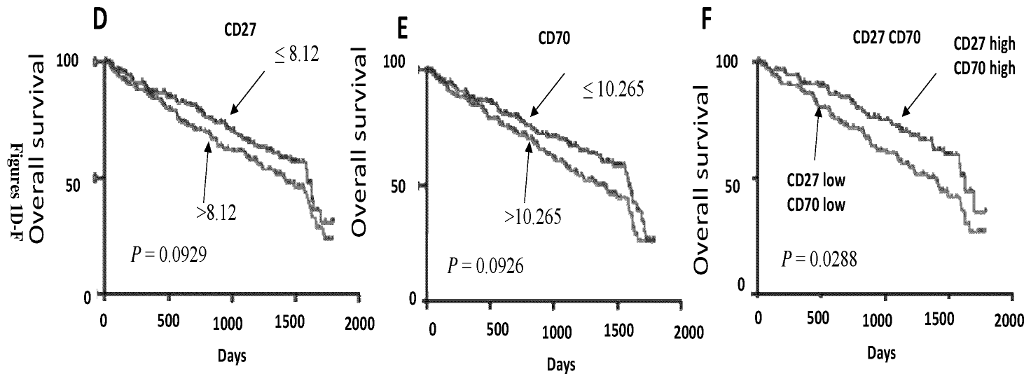

As result, we observed that CD27 was expressed on CD4⁺T cells and CD8⁺T cells while CD70 was expressed on tumor cells (PAX8). More importantly, interactions of CD27⁺T cells and CD70⁺ tumor cells can be seen in TME (data not shown). After phenotyping, CD27⁺ cells are composed of 65% CD4⁺CD27⁺T cells, 19% CD8⁺CD27⁺T cells and 16% other cells (data based on 7 samples) (data not shown), suggesting that CD27 is mostly expressed on T cells. To better measure the CD27−CD70 intracellular interaction, we did the spatial analysis by HALO software (data not shown). We defined CD27⁺ cells within 30 μm of CD70⁺ cells as “interacted CD27”, CD27⁺ cells and CD70⁺ cells with CD27⁺ cells around in a radius of 30 μm as “interacted CD70” (data not shown). Quantitative result of interacted CD27 and interacted CD70 was presented in FIG. 2. These results suggest that significant interaction exist between CD27 and CD70 positive cells. CD70⁺ cells seem to interact with more CD27⁺ cells than CD27⁺ cells with CD70⁺ cells. Since CD70⁺ is expressed by tumor cells, and CD27 is mainly expressed by T cells, the larger surface area occupied by the tumour cell compared to T cells may explain this result.

### Apoptosis of CD27⁺T Cells in ccRCC

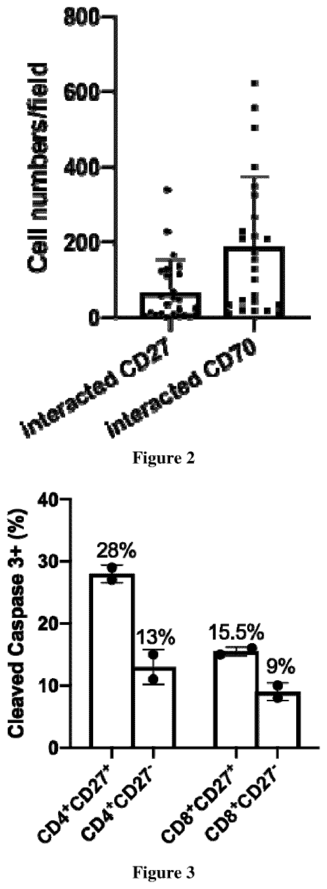

As the role of CD27−CD70 interaction is unclear in ccRCC, we next studied the phenotypic characteristic of CD27⁺T cells in tumors. Previous reports indicated that CD27-induced apoptosis was mediated by exposure to CD70 in ccRCC in vitro (Diegmann et al., 2006). We then asked whether CD70 highly expressing ccRCC induced the apoptosis of CD27⁺T cells in human. To confirm our hypothesis, we performed flow cytometry analysis on fresh tumors from 2 ccRCC patients. CD27 is expressed on CD4⁺ and CD8⁺T cells (data not shown). Caspase 3 is associated with cell apoptosis. Cleaved caspase 3 remains intact during apoptosis and can be detecting using substrate. In our study, cleaved caspase 3 is observed in TILs (data not shown). More importantly, we found a higher percentage of cleaved caspase 3 in CD27⁺T cells compared to CD27⁻T cells: CD4⁺CD27⁺T cells (28%) vs CD4⁺CD27⁻T cells (13%); CD8⁺CD27⁺T cells (15.5%) vs CD8⁺CD27⁻ T cells (9%) (FIG. 3). Despite the limited samples, it still suggests that CD27⁺T cells are more apoptotic than CD27⁻T cells, which may result from the CD27−CD70 interaction in tumor. However, this result has to be confirmed in a larger series of patients.

### CD27−CD70 Interaction In Situ Correlates with Elevated Levels of sCD27 in Peripheral Blood of ccRCC Patients

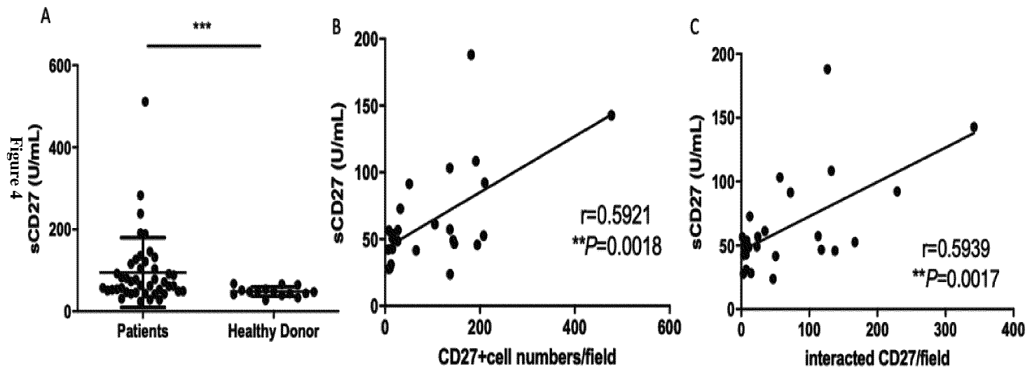

CD27 is known to be cleaved by a protease into its soluble form sCD27 on activated T cells upon CD27−CD70 interaction (Hintzen et al., 1991; Loenen et al., 1992a). Previous studies indicated that CD70-expressing glioma and RCC cell lines could induce the release of sCD27 from PBMCs (Ruf et al., 2015; Wischhusen et al., 2002). To investigate whether CD27−CD70 interaction in situ induced the release of sCD27, we first performed ELISA to measure plasma sCD27 from ccRCC patients. Elevated levels of sCD27 were observed in ccRCC patients (n=44) compared to healthy donors (n=15) (FIG. 4A). We then did the correlation analysis of sCD27 and CD27 cell counts in situ. sCD27 in peripheral significantly correlated with CD27⁺ cells in situ (FIG. 4B). More importantly, significant correlation was observed in interacted CD27⁺ cells in situ and sCD27 in peripheral (FIG. 4C), suggesting that elevated levels of sCD27 in the peripheral might come from the CD27−CD70 interaction in ccRCC.

### Apoptosis and Dysfunction of CD27+T cells in RCC

To confirm the phenotype of CD27+T cells, we sorted CD27+ and CD27−CD8+T cells and performed single-cell RNA sequencing (scRNA-seq) analysis from 4 renal tumor samples. CD8+CD27+ TILs had an expression profile of enriched genes associated with apoptosis from the Gene Ontology database composed of BAX, FASLG, BCL2L11, CYCS, FBXO32, LGALS1, PIK3R1, TERF1, TXNIP, CDKN2A. In addition, we confirmed the TRM phenotype of CD27+CD8+ T cells, as they more frequently express the ITGAE gene (CD103) and other transcription factors (PRDM1, RBPJ, ZNF683) associated with the TRM phenotype. Furthermore, single-cell RNAseq analysis also confirmed that CD27+CD8+T cells express markers of exhaustion (PDCD1, CTLA4, HAVCR2, LAG3, TIGIT, TNFRSF9, SIRPG, ICOS, LAYN, CXCL13, CD38, TOX) (data not shown). An increase of cytotoxicity-associated transcripts (GZMA, GZMB, GZMH, CTSC) was also observed in CD27+CD8+T cells. Finally, we showed that CD27−CD8+T cells more frequently express IL-2 and IL-7R associated with naive, central memory phenotype.

In conclusion, by using TCGA data, inventors found that the frequent expression pattern of CD27 and CD70 in ccRCC correlates with patient survival. They attempt to understand the role of CD27 and CD70 in ccRCC. In their study cohort, they observed the interaction of CD27+T cells and CD70+ tumor cells interaction in situ. The outcome of CD27−CD70 interaction may lead to CD27+ T cells apoptosis, which is suggested in the analysis of TILs by flow cytometry. Besides, plasma levels of sCD27 are elevated in ccRCC and correlate with CD27−CD70 interaction in situ. Our study demonstrate that CD27−CD70 interaction results in T cell dysfunction and the release of sCD27.

### REFERENCES

Throughout this application, various references describe the state of the art to which this invention pertains. The disclosures of these references are hereby incorporated by reference into the present disclosure.
Adam, P. J., Terrett, J. A., Steers, G., Stockwin, L., Loader, J. A., Fletcher, G. C., Lu, L. S., Leach, B. I., Mason, S., Stamps, A. C., et al. (2006). CD70 (TNFSF7) is expressed at high prevalence in renal cell carcinomas and is rapidly internalised on antibody binding. British journal of cancer 95, 298-306.
Ahrends, T., Ba̧bała, N., Xiao, Y., Yagita, H., van Eenennaam, H., and Borst, J. (2016). CD27 Agonism Plus PD-1 Blockade Recapitulates CD4+T-cell Help in Therapeutic Anticancer Vaccination. Cancer research 76, 2921-2931.
Akiba, H., Nakano, H., Nishinaka, S., Shindo, M., Kobata, T., Atsuta, M., Morimoto, C., Ware, C. F., Malinin, N. L., Wallach, D., et al. (1998). CD27, a member of the tumor necrosis factor receptor superfamily, activates NF-kappaB and stress-activated protein kinase/c-Jun N-terminal kinase via TRAF2, TRAF5, and NF-kappaB-inducing kinase. The Journal of biological chemistry 273, 13353-13358.
Arens, R., Nolte, M. A., Tesselaar, K., Heemskerk, B., Reedquist, K. A., van Lier, R. A. W., and van Oers, M. H. J. (2004). Signaling through CD70 regulates B cell activation and IgG production. J Immunol 173, 3901-3908.
Borst, J., Hendriks, J., and Xiao, Y. (2005). CD27 and CD70 in T cell and B cell activation. Current opinion in immunology 17, 275-281.
Brummelman, J., Mazza, E. M. C., Alvisi, G., Colombo, F. S., Grilli, A., Mikulak, J., Mavilio, D., Alloisio, M., Ferrari, F., Lopci, E., et al. (2018). High-dimensional single cell analysis identifies stem-like cytotoxic CD8(+) T cells infiltrating human tumors. The Journal of experimental medicine 215, 2520-2535.
Buchan, S. L., Fallatah, M., Thirdborough, S. M., Taraban, V. Y., Rogel, A., Thomas, L. J., Penfold, C. A., He, L.-Z., Curran, M. A., Keler, T., et al. (2018a). PD-1 Blockade and CD27 Stimulation Activate Distinct Transcriptional Programs That Synergize for CD8 T-Cell-Driven Antitumor Immunity. Clinical cancer research: an official journal of the American Association for Cancer Research 24, 2383-2394.
Buchan, S. L., Manzo, T., Flutter, B., Rogel, A., Edwards, N., Zhang, L., Sivakumaran, S., Ghorashian, S., Carpenter, B., Bennett, C. L., et al. (2015). OX40- and CD27-Mediated Costimulation Synergizes with Anti-PD-L1 Blockade by Forcing Exhausted CD8&lt;sup&gt;+&lt;/sup&gt; T Cells To Exit Quiescence. The Journal of Immunology 194, 125.
Buchan, S. L., Rogel, A., and Al-Shamkhani, A. (2018b). The immunobiology of CD27 and OX40 and their potential as targets for cancer immunotherapy. Blood 131, 39-48.
Burchill, M. A., Tamburini, B. A., and Kedl, R. M. (2015). T cells compete by cleaving cell surface CD27 and blocking access to CD70-bearing APCs. European journal of immunology 45, 3140-3149.
Camerini, D., Walz, G., Loenen, W. A., Borst, J., and Seed, B. (1991). The T cell activation antigen CD27 is a member of the nerve growth factor/tumor necrosis factor receptor gene family. J Immunol 147, 3165-3169.
Capitanio, U., Bensalah, K., Bex, A., Boorjian, S.A., Bray, F., Coleman, J., Gore, J. L., Sun, M., Wood, C., and Russo, P. (2019). Epidemiology of Renal Cell Carcinoma. Eur Urol 75, 74-84.
Ciccarelli, B. T., Yang, G., Hatjiharissi, E., Ioakimidis, L., Patterson, C. J., Manning, R. J., Xu, L., Liu, X., Tseng, H., Gong, P., et al. (2009). Soluble CD27 is a faithful marker of disease burden and is unaffected by the rituximab-induced IgM flare, as well as by plasmapheresis, in patients with Waldenstrom's macroglobulinemia. Clin Lymphoma Myeloma 9, 56-58.
Claus, C., Riether, C., Schürch, C., Matter, M. S., Hilmenyuk, T., and Ochsenbein, A. F. (2012). CD27 Signaling Increases the Frequency of Regulatory T Cells and Promotes Tumor Growth. Cancer research 72, 3664.
Dang, L. V. P., Nilsson, A., Ingelman-Sundberg, H., Cagigi, A., Gelinck, L. B. S., Titanji, K., De Milito, A., Grutzmeier, S., Hedlund, J., Kroon, F. P., et al. (2012). Soluble CD27 induces IgG production through activation of antigen-primed B cells. Journal of internal medicine 271, 282-293.
Diegmann, J., Junker, K., Loncarevic, I. F., Michel, S., Schimmel, B., and von Eggeling, F. (2006). Immune escape for renal cell carcinoma: CD70 mediates apoptosis in lymphocytes. Neoplasia (New York, NY) 8, 933-938.
Fridman, W. H., Zitvogel, L., Sautès-Fridman, C., and Kroemer, G. (2017). The immune contexture in cancer prognosis and treatment. Nature reviews Clinical oncology 14, 717-734.
García, P., De Heredia, A. B., Bellóon, T., Carpio, E., Llano, M., Caparrós, E., Aparicio, P., and López-Botet, M. (2004). Signalling via CD70, a member of the TNF family, regulates T cell functions. Journal of leukocyte biology 76, 263-270.
Giraldo, N. A., Becht, E., Pagès, F., Skliris, G., Verkarre, V., Vano, Y., Mejean, A., Saint-Aubert, N., Lacroix, L., Natario, I., et al. (2015). Orchestration and Prognostic Significance of Immune Checkpoints in the Microenvironment of Primary and Metastatic Renal Cell Cancer. Clinical cancer research: an official journal of the American Association for Cancer Research 21, 3031-3040.
Giraldo, N. A., Becht, E., Vano, Y., Petitprez, F., Lacroix, L., Validire, P., Sanchez-Salas, R., Ingels, A., Oudard, S., Moatti, A., et al. (2017). Tumor-Infiltrating and Peripheral Blood T-cell Immunophenotypes Predict Early Relapse in Localized Clear Cell Renal Cell Carcinoma. Clinical Cancer Research 23, 4416.
Gossage, L., Eisen, T., and Maher, E. R. (2015). VHL, the story of a tumour suppressor gene. Nature reviews Cancer 15, 55-64.
Goto, N., Tsurumi, H., Takemura, M., Kanemura, N., Kasahara, S., Hara, T., Yasuda, I., Shimizu, M., Yamada, T., Sawada, M., et al. (2012). Serum soluble CD27 level is associated with outcome in patients with diffuse large B-cell lymphoma treated with rituximab, cyclophosphamide, doxorubicin, vincristine and prednisolone. Leukemia & Lymphoma 53, 1494-1500.
Gudi, R., Barkinge, J., Hawkins, S., Chu, F., Manicassamy, S., Sun, Z., Duke-Cohan, J. S., and Prasad, K. V. S. (2006). Siva-1 negatively regulates NF-κB activity: effect on T-cell receptor-mediated activation-induced cell death (AICD). Oncogene 25, 3458-3462.
Han, B. K., Olsen, N. J., and Bottaro, A. (2016). The CD27−CD70 pathway and pathogenesis of autoimmune disease. Seminars in arthritis and rheumatism 45, 496-501.
He, L.-Z., Prostak, N., Thomas, L. J., Vitale, L., Weidlick, J., Crocker, A., Pilsmaker, C. D., Round, S. M., Tutt, A., Glennie, M. J., et al. (2013). Agonist anti-human CD27 monoclonal antibody induces T cell activation and tumor immunity in human CD27-transgenic mice. J Immunol 191, 4174-4183.
Hendriks, J., Gravestein, L. A., Tesselaar, K., van Lier, R. A., Schumacher, T. N., and Borst, J. (2000). CD27 is required for generation and long-term maintenance of T cell immunity. Nature immunology 1, 433-440.
Hendriks, J., Xiao, Y., and Borst, J. (2003). CD27 promotes survival of activated T cells and complements CD28 in generation and establishment of the effector T cell pool. The Journal of experimental medicine 198, 1369-1380.
Hintzen, R. Q., de Jong, R., Hack, C. E., Chamuleau, M., de Vries, E. F., ten Berge, I. J., Borst, J., and van Lier, R. A. (1991). A soluble form of the human T cell differentiation antigen CD27 is released after triggering of the TCR/CD3 complex. J Immunol 147, 29-35.
Hsieh, J. J., Purdue, M. P., Signoretti, S., Swanton, C., Albiges, L., Schmidinger, M., Heng, D. Y., Larkin, J., and Ficarra, V. (2017). Renal cell carcinoma. Nature reviews Disease primers 3, 17009.
Huang, J., Jochems, C., Anderson, A. M., Talaie, T., Jales, A., Madan, R. A., Hodge, J. W., Tsang, K. Y., Liewehr, D. J., Steinberg, S. M., et al. (2013). Soluble CD27-Pool in Humans May Contribute to T Cell Activation and Tumor Immunity. The Journal of Immunology 190, 6250.
Jilaveanu, L. B., Sznol, J., Aziz, S. A., Duchen, D., Kluger, H. M., and Camp, R. L. (2012). CD70 expression patterns in renal cell carcinoma. Human pathology 43, 1394-1399.
Kato, K., Chu, P., Takahashi, S., Hamada, H., and Kipps, T. J. (2007). Metalloprotease inhibitors block release of soluble CD27 and enhance the immune stimulatory activity of chronic lymphocytic leukemia cells. Exp Hematol 35, 434-442.
Kok, M., Bonfrer, J. M., Korse, C. M., de Jong, D., and Kersten, M. J. (2003). Serum soluble CD27, but not thymidine kinase, is an independent prognostic factor for outcome in indolent non-Hodgkin's lymphoma. Tumour biology: the journal of the International Society for Oncodevelopmental Biology and Medicine 24, 53-60.
Kuka, M., Munitic, I., Giardino Torchia, M. L., and Ashwell, J. D. (2013). CD70 is downregulated by interaction with CD27. J Immunol 191, 2282-2289.
Law, C.-L., Gordon, K. A., Toki, B. E., Yamane, A. K., Hering, M. A., Cerveny, C. G., Petroziello, J. M., Ryan, M. C., Smith, L., Simon, R., et al. (2006). Lymphocyte activation antigen CD70 expressed by renal cell carcinoma is a potential therapeutic target for anti-CD70 antibody-drug conjugates. Cancer research 66, 2328-2337.
Lawrence, M. S., Stojanov, P., Polak, P., Kryukov, G. V., Cibulskis, K., Sivachenko, A., Carter, S. L., Stewart, C., Mermel, C. H., Roberts, S. A., et al. (2013). Mutational heterogeneity in cancer and the search for new cancer-associated genes. Nature 499, 214-218.
Lens, S. M., Tesselaar, K., van Oers, M. H., and van Lier, R. A. (1998). Control of lymphocyte function through CD27−CD70 interactions. Semin Immunol 10, 491-499.
Loenen, W. A., De Vries, E., Gravestein, L. A., Hintzen, R. Q., Van Lier, R. A., and Borst, J. (1992a). The CD27 membrane receptor, a lymphocyte-specific member of the nerve growth factor receptor family, gives rise to a soluble form by protein processing that does not involve receptor endocytosis. European journal of immunology 22, 447-455.
Loenen, W. A., De Vries, E., Gravestein, L. A., Hintzen, R. Q., Van Lier, R. A., and Borst, J. (1992b). The CD27 membrane receptor, a lymphocyte-specific member of the nerve growth factor receptor family, gives rise to a soluble form by protein processing that does not involve receptor endocytosis. European journal of immunology 22, 447-455.
Mahnke, Y. D., Brodie, T. M., Sallusto, F., Roederer, M., and Lugli, E. (2013). The who's who of T-cell differentiation: human memory T-cell subsets. European journal of immunology 43, 2797-2809.
Matter, M., Odermatt, B., Yagita, H., Nuoffer, J. M., and Ochsenbein, A. F. (2006). Elimination of chronic viral infection by blocking CD27 signaling. The Journal of experimental medicine 203, 2145-2155.
Moon, H., Na, H.-Y., Chong, K. H., and Kim, T. J. (2006). P2X7 receptor-dependent ATP-induced shedding of CD27 in mouse lymphocytes. Immunol Lett 102.
Motzer, R. J., Escudier, B., McDermott, D. F., George, S., Hammers, H. J., Srinivas, S., Tykodi, S. S., Sosman, J. A., Procopio, G., Plimack, E. R., et al. (2015). Nivolumab versus Everolimus in Advanced Renal-Cell Carcinoma. The New England journal of medicine 373, 1803-1813.
Motzer, R. J., Penkov, K., Haanen, J., Rini, B., Albiges, L., Campbell, M. T., Venugopal, B., Kollmannsberger, C., Negrier, S., Uemura, M., et al. (2019). Avelumab plus Axitinib versus Sunitinib for Advanced Renal-Cell Carcinoma. The New England journal of medicine 380, 1103-1115.
Motzer, R. J., Tannir, N. M., McDermott, D. F., Arén Frontera, O., Melichar, B., Choueiri, T. K., Plimack, E. R., Barthélémy, P., Porta, C., George, S., et al. (2018). Nivolumab plus Ipilimumab versus Sunitinib in Advanced Renal-Cell Carcinoma. The New England journal of medicine 378, 1277-1290.
Nolte, M. A., van Olffen, R. W., van Gisbergen, K. P. J. M., and van Lier, R. A. W. (2009). Timing and tuning of CD27−CD70 interactions: the impact of signal strength in setting the balance between adaptive responses and immunopathology. Immunol Rev 229, 216-231.
Padanilam, B. J., Lewington, A. J., and Hammerman, M. R. (1998). Expression of CD27 and ischemia/reperfusion-induced expression of its ligand Siva in rat kidneys. Kidney Int 54, 1967-1975.
Prasad, K. V., Ao, Z., Yoon, Y., Wu, M. X., Rizk, M., Jacquot, S., and Schlossman, S. F. (1997). CD27, a member of the tumor necrosis factor receptor family, induces apoptosis and binds to Siva, a proapoptotic protein. Proceedings of the National Academy of Sciences of the United States of America 94, 6346-6351.
Py, B., Slomianny, C., Auberger, P., Petit, P. X., and Benichou, S. (2004). Siva-1 and an alternative splice form lacking the death domain, Siva-2, similarly induce apoptosis in T lymphocytes via a caspase-dependent mitochondrial pathway. J Immunol 172, 4008-4017.
Riether, C., Schürch, C. M., Bührer, E. D., Hinterbrandner, M., Huguenin, A.-L., Hoepner, S., Zlobec, I., Pabst, T., Radpour, R., and Ochsenbein, A. F. (2016). CD70/CD27 signaling promotes blast stemness and is a viable therapeutic target in acute myeloid leukemia. Journal of Experimental Medicine 214, 359-380.
Rini, B. I., Plimack, E. R., Stus, V., Gafanov, R., Hawkins, R., Nosov, D., Pouliot, F., Alekseev, B., Soulières, D., Melichar, B., et al. (2019). Pembrolizumab plus Axitinib versus Sunitinib for Advanced Renal-Cell Carcinoma. The New England journal of medicine 380, 1116-1127.
Ruf, M., Mittmann, C., Nowicka, A. M., Hartmann, A., Hermanns, T., Poyet, C., van den Broek, M., Sulser, T., Moch, H., and Schraml, P. (2015). pVHL/HIF-regulated CD70 expression is associated with infiltration of CD27+ lymphocytes and increased serum levels of soluble CD27 in clear cell renal cell carcinoma. Clinical cancer research: an official journal of the American Association for Cancer Research 21, 889-898.
Sakanishi, T., and Yagita, H. (2010). Anti-tumor effects of depleting and non-depleting anti-CD27 monoclonal antibodies in immune-competent mice. Biochem Biophys Res Commun 393, 829-835.
Şenbabaoğlu, Y., Gejman, R. S., Winer, A. G., Liu, M., Van Allen, E. M., de Velasco, G., Miao, D., Ostrovnaya, I., Drill, E., Luna, A., et al. (2016). Tumor immune microenvironment characterization in clear cell renal cell carcinoma identifies prognostic and immunotherapeutically relevant messenger RNA signatures. Genome biology 17, 231-231.
Stenzel, P. J., Schindeldecker, M., Tagscherer, K. E., Foersch, S., Herpel, E., Hohenfellner, M., Hatiboglu, G., Alt, J., Thomas, C., Haferkamp, A., et al. (2020). Prognostic and Predictive Value of Tumor-infiltrating Leukocytes and of Immune Checkpoint Molecules PD1 and PDL1 in Clear Cell Renal Cell Carcinoma. Transl Oncol 13, 336-345.
Tesselaar, K., Arens, R., van Schijndel, G. M. W., Baars, P. A., van der Valk, M. A., Borst, J., van Oers, M. H. J., and van Lier, R. A. W. (2003). Lethal T cell immunodeficiency induced by chronic costimulation via CD27−CD70 interactions. Nature immunology 4, 49-54.
van Oers, M. H., Pals, S. T., Evers, L. M., van der Schoot, C. E., Koopman, G., Bonfrer, J. M., Hintzen, R. Q., von dem Borne, A. E., and van Lier, R. A. (1993). Expression and release of CD27 in human B-cell malignancies. Blood 82, 3430-3436.
Wajant, H. (2016). Therapeutic targeting of CD70 and CD27. Expert Opinion on Therapeutic Targets 20, 959-973.
Wang, Q. J., Hanada, K.-I., Robbins, P. F., Li, Y. F., and Yang, J. C. (2012). Distinctive features of the differentiated phenotype and infiltration of tumor-reactive lymphocytes in clear cell renal cell carcinoma. Cancer research 72, 6119-6129.
Wischhusen, J., Jung, G., Radovanovic, I., Beier, C., Steinbach, J. P., Rimner, A., Huang, H., Schulz, J. B., Ohgaki, H., Aguzzi, A., et al. (2002). Identification of CD70-mediated apoptosis of immune effector cells as a novel immune escape pathway of human glioblastoma. Cancer research 62, 2592-2599.
Yoon, Y., Ao, Z., Cheng, Y., Schlossman, S. F., and Prasad, K. V. S. (1999). Murine Siva-1 and Siva-2, alternate splice forms of the mouse Siva gene, both bind to CD27 but differentially transduce apoptosis. Oncogene 18, 7174-7179.

1. (canceled)

2. (canceled)

3. A method for predicting whether a subject suffers from or is susceptible to suffer from a cancer and/or metastatic cancer expressing CD70 and treating the subject, comprising: i) determining the level of soluble CD27 (sCD27) in a biological sample from the subject; ii) determining that the level of sCD27 in the biological sample is higher than a corresponding predetermined reference value; and iii) treating the subject determined to have a level of sCD27 that is higher than its corresponding predetermined reference value with a therapeutically effective amount of an inhibitor of interaction of CD27/CD70.

4. The method of claim 3, wherein the biological sample is a plasma sample.

5. A method for treating cancer and/or metastatic cancer in a subject in need thereof comprising administering to said subject a therapeutically effective amount of an inhibitor of interaction of CD27/CD70.

6. The method according to claim 5 further comprising, before the step of administering, a step of determining the interaction between CD27 and CD70 by i) determining the level of soluble CD27 (sCD27) in a biological sample from the subject and ii) determining that the level of sCD27 is higher than a corresponding predetermined reference value.

7. The method according to claim 5, wherein said inhibitor of interaction of CD27/CD70 is an inhibitor of CD27.

8. The method according to claim 5, wherein said inhibitor of interaction of CD27/CD70 is an anti-CD27 neutralizing monoclonal antibody.

9. The method according to claim 5, wherein said inhibitor of interaction of CD27/CD70 is an inhibitor of CD70.

10. The method according to claim 5, wherein said inhibitor of interaction of CD27/CD70 is an anti-CD70 neutralizing monoclonal antibody.

11. A method of preventing and/or treating cancer and/or metastatic cancer in a subject in need thereof, comprising, administering to the subject a therapeutically effective amount of an inhibitor of interaction of CD27/CD70 and a classical cancer treatment.

12. The method according to claim 11, wherein the classical cancer treatment is an immune checkpoint inhibitor.

13. The method according to claim 12, wherein the immune checkpoint inhibitor is an anti-PD-1 antibody, an anti-PD-L1 antibody or an anti-PD-L2 antibody.

14. A kit or device for performing the method of claim 1, comprising means for determining the level of the soluble CD27 in a biological sample.


SEQUENCE LISTING (6 sequences)

SEQ ID NO: 1 - DNA, 911 bp, Homo sapiens
agagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtggtggcaggacaagagaaggcattgagctttttctttcattttcctattaaaaaatacaaaaatca

SEQ ID NO: 2 - DNA, 2427 bp, Homo sapiens
agagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaagggctttttggattttggaactggggactcaaagtcaagtgcttcttacggcatttaatatggactgcacactgttttatcccattaactcagctcgtgttcatgcaagccctacaaagctggaggaatcatcattgttcccatttcacagatgaggaaaacagaggcgtaaaccgttgagtcatctgtccaaagttactggttttgtaacagctggaacctcagtatttaacagttgaggaaaatagtggcagaaaacctgagtcacaagcccgaggcaattcatctacatagtagtggactcaggattccaagccagataatcccgcatcttgcatccatgatctcacttctagaaggggaagtgagaggagtggtggggtgtcccagctgagggagggaaaatgagacccatgcgccacccgccctcacaaatctagccctgcagcccctctccaaccttacctggactcagaaccatgcatctggaaaagccacagacactcgacgccagcctgtgaaagcagccgggagggaggctgtatcctgcaaagccacagggaaggggctgcccaagaccatgggaacccaccacttacatcagcgtgacctggatgtgagacacagagtcaaaggagattatctggcagctttaagattggactgcctgactattcacaatagcaaagacttggaaccaacccaaatgtccatcaatgatagactggattaagaaaatgtggcacatatacaccatggaatactatgcagccataaaaacggatgagttcatgtccttcatagggacatggatgaagctggaaaccatcattctgagcaaactattgcaaggacagaaaaccaaacaccgcatgttctcactcataggtggaaattgaacaatgagaatacttggacacagggaggagaacatcacacaccggggcctgtcgtggagtgtggagtcgggggagggggggagggatagcattaggagaaatacctaatgtaaatgacaaattaatgggtgcagtaaaccaacacggcacatatatacatatgtaacaaatctgcacgttgtgcacatgtaccctagaacttaaagtataattaaaaaaaaaaaaaaaaaaagatttgactgcctggccaggcacagtggctcatgcctataatcctaacactttgggaggccgaggcgagcagattgcctgagctgaagagttcgagaccagcctgggcaatatggtgaaaccccgtttctactaaaatacaaaaaaaaaaaaattagccaggcgtggcagcgtgcgcctgtagtccccgcgacttggaggctgatgcaggagaatcgctttaacccgggaggcagaggttgcagtgagccgagatcgagccactgtactccagactgggtgacagagcgacactctgtctcaaaaaaaaaaaaaagatttaactgccccactggattttggaattgcaggggaccttcagccccttcgtttgtccagtttctcccatttggaatgggtatatccaacgcctgtaccccattgtatataggaagtaactaacttgcttttgattttacaggctcacagaagggacgtgccttgtctcagatgagattttggactgtggacttttgagttaatgctgaaatgagttaagactttgggggatagttaggaaggcatgactagtttgaggacattatactcaatgaaataagccagtcaaaaaggaaaaatatgtattgtataaactaacttacattaatctaccacataggattaaaactgttcacaggaatataaaatagaattttttagata

SEQ ID NO: 3 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 4 - protein, 208 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGLFGFWNWGLKVKCFLRHLIWTAHCFIPLTQLVFMQALQSWRNHHCSHFTDEENRGVNR

SEQ ID NO: 5 - DNA, 1245 bp, Homo sapiens
cttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcattacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccacc

SEQ ID NO: 6 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP